(19) United States
(12) Patent Application Publication
(10) Pub. No.: US 2011/0262526 A1
(43) Pub. Date: Oct. 27, 2011

(54) METHOD OF INDUCING AN ANTI-VIRAL IMMUNE RESPONSE

(76) Inventor: Barton F. Haynes, Durham, NC (US)

(21) Appl. No.: 12/737,984

(22) PCT Filed: Sep. 8, 2009
(86) PCT No.: PCT/US2009/005024

§ 371 Date: Jun. 23, 2011

Related U.S. Application Data
(60) Provisional application No. 61/136,448, filed on Sep. 5, 2008.
(60) Provisional application No. 61/136,734, filed on Sep. 29, 2008.

(51) Int. Cl. A61K 9/127; A61P 31/18; A61P 37/04; A61K 39/21

(52) U.S. Cl. 424/450; 424/208.1

(57) ABSTRACT

The present invention relates to a method of inducing an anti-viral immune response. The method comprises administering to a patient in need thereof an antigen that induces the production of antibodies that, upon binding to a cell surface target, result in the production of chemokines that inhibit viral infection.

This application claims priority from U.S. Provisional Appln. No. 61/136,448, filed Sep. 5, 2008, and U.S. Provisional Appln. No. 61/136,734, filed Sep. 29, 2008, the entire contents of which are hereby incorporated by reference.

This invention was made with government support under Grant No. UO1 AI067854 awarded by the National Institutes of Health. The government has certain rights in the invention.

### TECHNICAL FIELD

The present invention relates to a method of inducing an anti-viral immune response. The method comprises administering to a patient in need thereof an immunogen that induces the production of antibodies which, upon binding to a cell surface target, result in the production of cytokines (e.g., chemokines) that inhibit viral infection.

### BACKGROUND

A major challenge for HIV and other viral vaccine development (e.g., hepatitis C vaccine development) is the need for induction of a rapid anti-viral immune response (Gasper-Smith et al, J. Virol. 82:7700-7710 (2008)). The innate immune response has as one of its attributes rapid immune response induction following pathogen transmission. Innate immune responses to transmitted pathogens can arise in hours to days. However, innate immunity lacks immunologic memory and, thus, cannot be primed by pathogens or a vaccine for an accelerated or enhanced response (Haynes et al, Introduction to the Immune System in “Harrisons Principles of Internal Medicine” Chapter 308: 17^{th} Edition, Fauci, Kasper, Hauser, Longo, Jameson, Loscalzo (Editors), McGraw Hill, New York (2008)). In contrast, the adaptive or acquired immune system has the capacity of immune memory by virtue of B cell receptor (BCR) and T cell receptor (TCR) rearranging genes (Haynes et al, Introduction to the Immune System in “Harrisons Principles of Internal Medicine” Chapter 308: 17^{th} Edition, Fauci, Kasper, Hauser, Longo, Jameson, Loscalzo (Editors), McGraw Hill, New York (2008)). However, adaptive immune responses can take days to weeks to arise. The adaptive T and B cell arms of the immune system can make antigen-specific responses to specific pathogens. Since the innate immune system has no antigen receptor rearranging genes, it cannot make responses that are highly specific to a given pathogen.

Traditional vaccines (such as measles, mumps and rubella vaccines) rely on induction of memory adaptive T and B cell responses for their success as preventive vaccines (Plotkin, Clin. Infect. Dis. 47:401-409 (2008)). For the pathogens that these successful vaccines protect against, there is no need for a rapid memory response (e.g., within hours) since the organisms are relatively slow to develop and do not insert their genetic material into the host genome to form a reservoir of pathogen that is protected from the immune system.

HIV-1 has a very short “eclipse” phase, that is, that period of time from transmission to appearance of virus in the plasma (Gasper-Smith et al, J. Virol. 82:770-7710 (2008)). Further, HIV-1 establishes a latent pool of infected CD4 T cells, likely within the first week of infection—such latent pools of virus are invisible to the immune system (Shen et al, Aller. & Clin. Immunol. 122:22-28 (2008)).

HIV-1 utilizes a chemokine receptor as a co-receptor, most commonly CCR5 by the transmitted virus (Keele et al, Proc. Natl. Acad. Sci. 105:7552-7557 (2008)) or CXCR4 by chronic viruses (Kinter et al, Proc. Natl. Acad. Sci. 93:14076-14081 (1996), Rubbert et al, AIDS Res. Hum. Retrovirol. 13:63-69 (1997), Kinter et al, Immunol. Rev. 177:88-98 (2000)). The ligands for CCR5 are the chemokines macrophage inflammatory protein-1α (MIP-1α), MIP-1β and RANTES (Kinter et al, Immunol. Rev. 177:88-98 (2000)). These chemokines, when present and produced by CD8+ T cells or monocytes (or other cells of the myeloid lineage such as tissue macrophages, dendritic cells or cells of non-myeloid lineage such as but not limited to epithelial cells), can have profound blocking effects on infectivity of CCR5-utilizing HIV strains (Kinter et al, Immunol. Rev. 177:88-98 (2000)). Similarly, SDF-1 is a ligand for CXCR4 and can inhibit CXCR4-utilizing HIV strains (Kinter et al, Immunol. Rev. 177:88-98 (2000)).

Ligation of CD40 with CD40 ligands induces production of chemokines such as IL-8, MIP-1α, MIP-1β and RANTES, as well as production of cytokines such as TNF-α, interleukin (IL)-12, IL-1, IL-10, and IL-15 (Banchereau et al, Annu. Rev. Immunol. 12:881-922 (1994), Chess et al, Therapeutic Immunology 2^{nd} edition, pgs. 441-456 (2001), Brodeur et al, Immunity 18:837-848 (2003), di Marzio et al, Cytokine 12:1489-1495 (2000), Chougnet et al, J. Immunol. 163:1666-1673 (1999)). The interaction between CD40 and its cognate ligand, CD40L (CD154), is critical for a productive immune response (Ellmark et al, AIDS Res. Hum. Retrovirol. 24:367-373 (2008), Abayneh et al, AIDS Res. Hum. Retrovirol. 24:447-452 (2008), Munch et al, Cell 129:263-275 (2007)). Other molecules, such as c4b-binding protein, also bind to CD40 (Schonbeck et al, Cell Mol. Life Sci. 58:4-43 (2001)). CD40 on monocytes, macrophages and dendritic cells binds to CD40 ligand on T cells and this interaction is central in the mediation of T cell antigen recognition, induction of T cell help, and induction of B cell immunoglobulin class switching. Humans with mutations in either the CD40 molecule or the CD40 ligand molecule have an inability to class switch immunoglobulins called the Hyper IgM Syndrome (Kiener et al, J. Immunol. 155:4917-4925 (1995)).

Ellmark and colleagues have isolated a series of anti-CD40 antibodies from a phage displayed library derived from a HIV uninfected subject (Ellmark et al, AIDS Res. Hum Retrovirol. 24:367-373 (2008)). They have shown that one of these human CD40 antibodies, B44, is capable of triggering B cells and monocytes to make chemokines, and can activate B cell division, The B44 monoclonal antibody (mAb) does not interfere with cognate CD40-CD40 ligand interaction towards mediating normal T cell—antigen presenting cell interactions (Ellmark et al, AIDS Res. Hum. Retrovirol. 24:367-373 (2008)). Ellmark and colleagues have also shown that the CD40 mAb, B44, inhibits HIV infectivity of the MonoMac monocyte cell line. Moreover, Abayneh et al have shown that the mechanism of CD40 mAb B44 inhibition of infection of MonoMac cells is by induction of chemokines from the cell line that inhibits CCR5 tropic viruses (Ellmark et al, AIDS Res. Hum. Retrovirol. 24:367-373 (2008), Abayneh et al, AIDS Res. Hum. Retrovirol. 24:447-452 (2008)). Thus, this work shows that CD40 on various cell types, including but not limited to monocytes, macrophages, dendritic cells and B cells, when ligated by an antibody, can trigger the induction of CCR5-binding chemokines (MIP-1α, MIP-1β, and Rantes). Ellmark et al has proposed that B44 mAb may be a therapeutic antibody suitable for treating active HIV-1 infection (Ellmark et al, AIDS Res. Hum. Retrovirol. 24:367-373 (2008)).

The present invention provides a vaccine that can induce memory in innate anti-viral immune responses so that a response to viral transmission and challenge occurs within hours of viral infection (Haynes et al, J. Aller. & Clin. Immunol. 122:3-9 (2008), Gasper-Smith et al, J. Virol. 82:7700-7710 (2008)). In most current successful vaccines, an adjuvant triggers the innate immune system to recruit the adaptive immune system to make an anti-viral immune response that takes several weeks to mature; when the infectious agent challenges the vaccinated subject, a more rapid adaptive (T and B cell response) occurs that takes days to weeks to occur. Proposed HIV-1 vaccines have largely been designed on the basis of this same strategy and, thus, require sequential activation of the innate and then the adaptive immune response for effectiveness.

The present invention is based on the recognition that a novel vaccine development strategy for fast-acting infections that quickly induce massive immune system dysfunction (e.g., HIV-1) is to have a preexisting adaptive B response present that an HIV-1 transmitted virus will boost immediately upon host contact with the transmitted virus, followed by the antibody recruiting an immediate and robust innate immune response. The present invention utilizes induction of antibodies against certain self molecules (for example, the CD40 molecule or cell surface lipids) with immunologic memory in the antibody response, and has, as the effector arm of the vaccine-induced immune response, the induction of innate anti-viral cytokines (e.g., chemokines such as MIP-1α, MIP-1β and RANTES)—that is, just the reverse of current vaccines. By this unique joining of the slow adaptive (memory-containing) B cell response (that is vaccine-induced prior to viral infection) with the fast innate cytokine response that is boosted following viral (e.g., HIV-1) infection, a non-pathogenic host antibody response can synergize with an innate anti-viral response to trigger a protective anti-viral cytokine response. Thus, in effect, the approach disclosed herein provides induced “innate memory” for a vaccine-primed anti-HIV-1 response within hours of infection by HIV-1.

### SUMMARY OF THE INVENTION

The present invention relates generally to a novel anti-viral (e.g., anti-HIV-1) vaccine strategy that encompasses a method of inducing a rapid anti-viral immune response. More specifically, the invention relates to a method of inducing an anti-viral immune response that comprises administering to a patient in need thereof an immunogen that induces the production of host antibodies which, upon binding to a cell surface target, result in the production and release of cytokines (e.g., chemokines) in an amount sufficient to inhibit viral infection.

Objects and advantages of the present invention will be clear from the description that follows.

### BRIEF DESCRIPTION OF THE DRAWINGS

FIG. 1. Effect of antibodies on chemokine expression levels in PBMC induced by anti-lipid antibodies in the presence and absence of HIV-1 infection.

FIG. 2. HIV-1 inhibition activity assayed. Pre-incubation of mAbs with either virus or cells.

FIG. 3. HIV-1 inhibiting activity of P1, IS4 and CL1 is inhibited by lipids such as cardiolipin and DOPE.

FIG. 4. Sequence comparison of rhesus, mouse and human CD40. The amino acids underlined are the acid amino acids of human CD40 (CD84, E114 and E117) that interface with the basic amino acids of CD40L.

FIG. 5. Immunogen design for induction of anti-CD40 antibodies.

FIG. 6. Blocking of CL1 HIV-1 inhibition activity by anti-chemokine antibodies.

FIG. 7. Mechanism of action of anti-lipid antibody inhibition of HIV-1 infectivity: a novel strategy for HIV-1 vaccine induction of innate memory responses against R5-transmitted viruses.

### DETAILED DESCRIPTION OF THE INVENTION

The present invention results, at least in part, from the realization that certain B cell antibodies can confer upon the innate immune system the ability to make high levels of cytokines (e.g., chemokines) in the presence of virus (e.g., HIV-1). The vaccination method of the instant invention takes advantage of the ability of certain immunogens to induce production of antibodies that, both alone and in the presence of virus (e.g., HIV-1), induce a rapid innate anti-viral immune response. Moreover, infection with the virus (e.g., HIV-1) can induce anti-lipid antibodies with this anti-viral effect, thus providing a booster effect for the innate chemokine-triggering antibody response. The invention can use the memory of the adaptive B cell immune response to trigger a rapid anti-viral innate cytokine (e.g., chemokine) response. In accordance with the invention, certain self molecules, when bound by induced antibodies, can trigger anti-viral innate substances (e.g., chemokines), particularly in the presence of the pathogen, and, moreover, the pathogen can induce a boost of the anti-lipid antibody as well.

The present invention relates, in one embodiment, to a method of inhibiting infection of susceptible cells (e.g., T-cells) of a subject by a CCR5-tropic strain of HIV-1. The method comprises administering to the subject an immunogen that induces the production of antibodies that bind to cells of the subject that: i) produce CCR5-binding chemokines, and ii) have on their surface an antigen recognized by the antibodies. Binding of the antibodies to the cell surface antigen induces the production by such cells of the CCR5-binding chemokines. In the presence, or absence, of the CCR5-tropic strain of HIV-1, the level of chemokines produced is sufficient to inhibit infection of the subject's T-cells. Thus, in accordance with the invention, an antibody that induces the production and release of CCR5-binding chemokines can be used to induce an anti-HIV innate (chemokine) response with memory (derived from the antibody response primed by administration of the immunogen).

Suitable cell surface target antigens include any molecule on the surface of a monocyte, macrophage or dendritic cell (or on the surface of any other cell, such as an epithelial cell, that can produce CCR5-binding chemokines) that has the capacity, when bound by an antibody, to trigger the production of CCR5-binding chemokines. Preferred targets include surface lipids of the cell lipid bilayer, such as phosphatidylserine (PS) and phosphatidylethanolamine (PE).

Suitable forms of immunogens capable of inducing the desired anti-lipid antibodies include PS- and PE-containing liposomes adjuvanted in monophosphoryl lipid A, TLR-7 or TLR-9-containing adjuvants (see, for example, PCT/US2008/004709). Additional polymorphic forms of lipids can also be used such as hexagonal II forms of PS or PE (Rauch et al, Proc. Natl. Acad. Sci. 87:4112-4114 (1990)). Also suitable for use in inducing anti-lipid antibodies are killed syphilis spirochetes (Wong et al, B. J. Vener. Dis. 59:220-224 (1983), Jones et al, Br. J. Vener. Dis. 52:9-17 (1976)). It will be appreciated that it is preferred that the induced antibodies be non-pathogenic. Criteria for pathogenicity of anti-lipid antibodies include their dependence on the β-2 glycoprotein 1 molecule as a cofactor for binding to lipids and their ability to cause thrombosis in the pinched ear lobe of a mouse (Zhao et al, Arth. Rheu. 42:2132-2138 (1999)). Thus, characteristics of preferred anti-lipid antibodies include: no binding to β-2 glycoprotein 1 and no thrombosis in a host that produces the antibody at physiologic concentrations. Alving et al have used various lipids to induce a variety of anti-lipid antibodies (Schuster et al, J. Immunol. 122:900-905 (1979)); most are not pathogenic, including anti-lipid antibodies made in syphilis and other infectious diseases (Alving, J. Lip. Res. 16:157-166 (2006)).

Immunogens suitable for use in the invention include highly purified anionic lipids, such as CL, PS, DOPE and PE, and other lipids from, for example, Avanti Polar Lipids or VDRL antigen (such as from Lee Laboratories) or killed Treponema pallidum (such as from Lee Laboratories).

The anti-lipid antibody-inducing immunogens can be administered intramuscularly (IM), subcutaneously or intravenously (IV). Optimal immunogen doses suitable for use in human subjects can be readily determined by one skilled in the art and can vary, for example, with the immunogen and with the subject. Immunogen doses can be, for example, about 100 μg of purified lipids, about 10⁵ to about 10⁶ killed T. Palidum organisms, about 100 μg of VDRL lipids, or about 200 μg of CL and/or PS liposomes. The immunogens can be administered by a mucosal route using cholera toxin (CT) or an inactivated version of CT or another mucosal adjuvant such as IL-1 (U.S. Pat. Nos. 7,041,294 and 6,270,758) for induction of anti-lipid antibodies at mucosal sites. Again, optimal doses suitable for use in humans can be readily determined by one skilled in the art. Examples of dose ranges include 10-100 μg IL-1 intranasally (IN), and 5-25 μg of inactivated CT IN.

A further preferred cell surface target antigen is the CD40 molecule. CD40 is a member of the tumor necrosis factor (TNF) receptor superfamily. It is expressed by a wide variety of cells, such as B cells, macrophages, dendritic cells (DC), keratinocytes, endothelial cells, thymic epithelial cells, fibroblasts, and tumor cells.

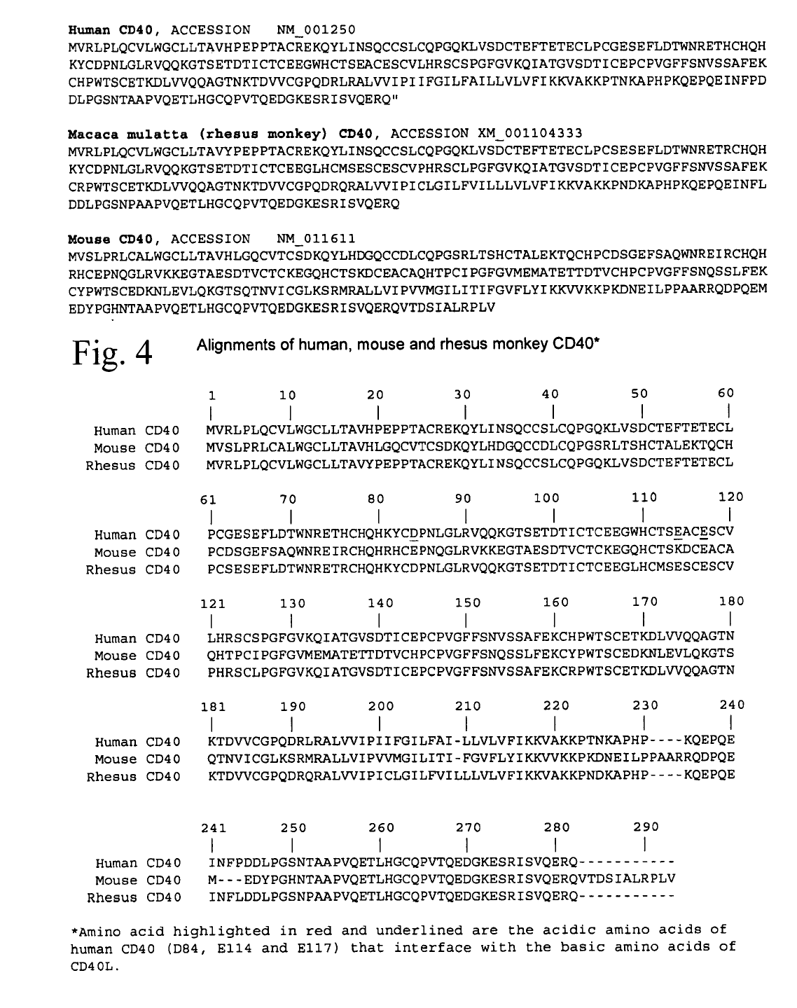

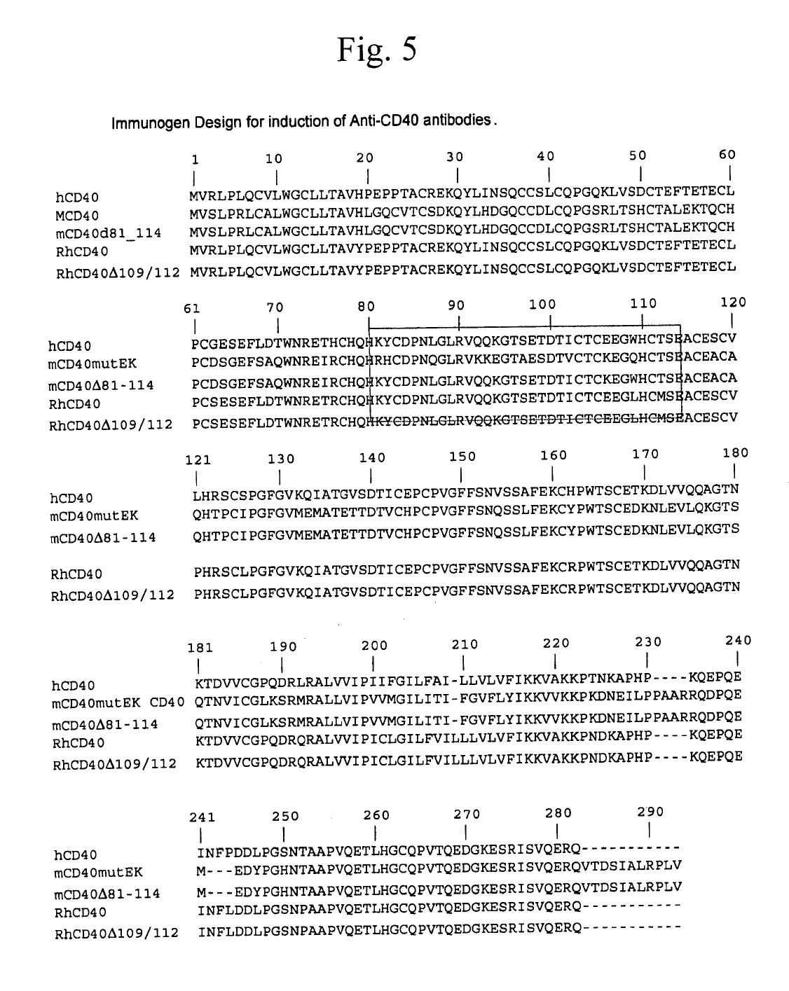

Suitable immunogens capable of inducing anti-CD40 antibodies include free or deriviatized human CD40 (derivatized by a carrier such as tetanus toxoid or keyhole limpet hemocyanin) or free or derivatized rhesus CD40 or other species of CD40 that is similar, but not identical, to human CD40 and thus optimally recognized to induce the anti-CD40 antibodies in human patients. Suitable immunogens include recombinant protein or DNA from rhesus monkey, guinea pig or mouse CD40 which, when immunized into humans, raise an anti-CD40 antibody that does not bind to the CD40Ligand binding site on CD40 but to other sites on CD40 to trigger an R5 chemokine release from the CD40+ cell. Sequence alignments of human, mouse and rhesus CD40 molecules are shown in FIG. 4. It has been determined that the acidic amino acids at positions D84, E114 and E117 of human CD40 interface with the basic amino acids of CD40L (Singh et al, Protein Science 7:1124-1135 (1998)). The amino acids in the CD40L interface region (D84, E114 and E117) of rhesus monkey are identical to those in human CD40 (FIG. 4), while only 2 amino acids (amino acids at positions 109 and 112) in the spanning region of the interface (amino acid position 81 to 114) are different between rhesus monkey and human CD40 proteins (FIG. 5). Amino acids in the corresponding spanning region of the interface are substantially different between mouse CD40 and human CD40 (FIGS. 4 and 5). Since the binding of CD40L to CD40 is critical to the overall physiological function of CD40 and induction of antibodies that bind to the binding site region of CD40L are not preferred, 2 mutant mouse CD40 constructs and one mutant rhesus monkey CD40 construct have been designed to reflect of the CD40L interface region more close to or identical to human CD40 (FIG. 5).

The immunogen used to induce anti-CD40 antibodies can be a protein, such as described in FIG. 5. Alternatively, the immunogen can be a nucleic acid (e.g., DNA) encoding such a protein. The nucleic acid can be administered as naked DNA or it can be present in a vector. The invention includes the proteins and encoding sequences, and constructs comprising the encoding sequences and a vector, and methods of using same to induce antibodies in a subject (human). Suitable vectors include BCG or other recombinant mycobacteria, recombinant pox virus vector, such as NYVAC, recombinant adenovirus vector, or in a flavi virus vector such as the yellow fever vaccine. The nucleic acid can be operably linked to a promoter. The protein or encoding nucleic acid can be administered, for example, IM, or subcutaneously. Optimal doses suitable for use in humans can be readily determined by one skilled in the art. Examples of dose ranges include rAd=about 10⁸ pfu to about 10⁹ pfu, protein=about 100-200 μg/dose IM, DNA=about 1-5 mg of DNA. The protein or encoding sequence can also be administered via a mucosal route. In the latter case, the protein or encoding nucleic acid can be administered with cholera toxin or an attenuated version of CT or with another mucosal adjuvant such as IL-1 (U.S. Pat. Nos. 6,270,758 or 7,041,294). Again, optimal doses suitable for use in humans can be readily determined by one skilled in the art. Examples of dose ranges include 10-100 μg IL-1 intranasally (IN), and 5-25 μg of inactivated CT IN.

The invention is described in detail above with reference to the production of antibodies specific for host cell surface targets (e.g., lipids and CD40). However, the invention includes the administration of any immunogen that results in the production of antibodies that, upon binding to target molecules, elicit an anti-HIV chemokine response. For example, suitable for use are immunogens that induce the production of antibodies that bind to a molecule on the virion or immunogens that induce the production of antibodies that bind to a molecule on the virion and a molecule on a host cell surface, where binding of such antibodies to target molecules induces anti-HIV chemokines within hours to days of transmission. Examples of such suitable immunogens include those that result in the production of m43- or m9-type antibodies (that is, antibodies having the specificity of m43 or m9) (Choudhry et al, Biochim. Biophys. Res. Comm. 348:1107-1115 (2006), Zhang et al, Current Pharm. Design. 13:203-212 (2007)). (See also WO 2006/050219.)

Antibodies (e.g., anti-lipid antibodies) produced in accordance with the present method can induce therapeutic levels of chemokines. In the presence of HIV-1 virions, the antibodies can induce more of the CCR5-binding chemokines (e.g., in excess of 20,000 μg/ml in vitro). This is important to the success and to the safety of the strategy. That the highest levels of chemokines occur in the presence of the antibody plus HIV-1 also imparts an antigen specificity to the response that ordinarily is not present in the innate immune system.

While the invention has been described in detail with reference to CCR5-tropic HIV-1 infection, it will be appreciated from a reading of the disclosure that a similar strategy can be adopted for CXCR4-utilizing HIV-1 strains. In the case of CXCR4 strains, the immunogen administered can be one designed to induce the production of antibodies that trigger the release of SDF-1 from target cells. Similarly, the present strategy can be adopted for Hepatitis B and C infections. Here, the immunogen administered can be one that induces the production of α-interferon or other protective cytokine.

Chemokines are not the only type of anti-viral molecules that an antibody can be designed to induce. Innate system small molecules such, as the VIRIP fragment of α-1 anti trypsin (Zhu et al, British Journal of Haematology 105:102-109 (1999)), soluble amyloid A, and β-defensins all have anti-viral (e.g., anti-HIV) activity and the induction of these molecules in a similar manner to induction of the CCR5-binding chemokines can be expected to have a salutary effect on preventing or treating HIV infection.

In a further embodiment, the present invention relates to compositions (e.g., pharmaceutical compositions) comprising an immunogen as described above and a carrier. Suitable carriers include, for example, sterile saline or buffer. The composition can be in a form suitable for injection or topical application, e.g., to a mucosal surface.

Certain aspects of the invention can be described in greater detail in the non-limiting Examples that follow. (See also Lin et al, Arth. Rheu. 56:1638-1647 (2007), Zhu et al, Br. J. Haem. 105:102-109 (1999), Lin et al, Arth. Rheu. 56:1638-1647 (2007), U.S. Prov. Appln. 61/136,448, filed Sep. 5, 2008.)

### Example 1

It has been postulated previously that the reason that subjects with autoimmune disease have a lower incidence of HIV-1 infection is related to tolerance defects in autoimmune disease subjects. It has been further postulated that these defects can lead to the production of certain types of antibodies that are capable of preventing infection of human cells by HIV-1 (Haynes et al, Human Antibodies 14:59-67 (2005), Haynes et al, Science 308:1906 (2005)). During the study of anti-lipid antibodies derived from humans with autoimmune disease, such as systemic lupus erythematosus (mAb CL1) and anti-phospholipid syndrome (P1 and IS4), it has been found that these antibodies prevent HIV-1 infection in a human peripheral blood mononuclear cell (PBMC) assay (Bures et al, AIDS Res. Hum. Retroviruses 16:2019-2035 (2000), Montefiori et al, J. Virol. 72:1886-1893 (1998), Montefiori et al, J. Infect. Dis. 173:60-67 (1996)) (Table 1) but not in the CD4-, CCR5 and CXCR4-transfected epithelial cell TZMBL pseudovirus assay (Wei et al, Nature 422:307-312 (2003), Derdeyn et al, J. Virol. 74:8358-8367 (2000), Li et al, J. Virol. 79:10108-10125 (2005), Montefiori, DC pp. 12.11.1-12.11.15, In Current Protocols in Immunology (2004)) (Table 2) (see PCT/US2008/004709).

In the PBMC assay, the lipid antibodies are found to neutralize only CCR5-utilizing strains of HIV, not CXCR4-utilizing strains (Table 3). Thus, if these anti-lipid antibodies can be induced, they can be protective against HIV-1 (see PCT/US2008/004709).

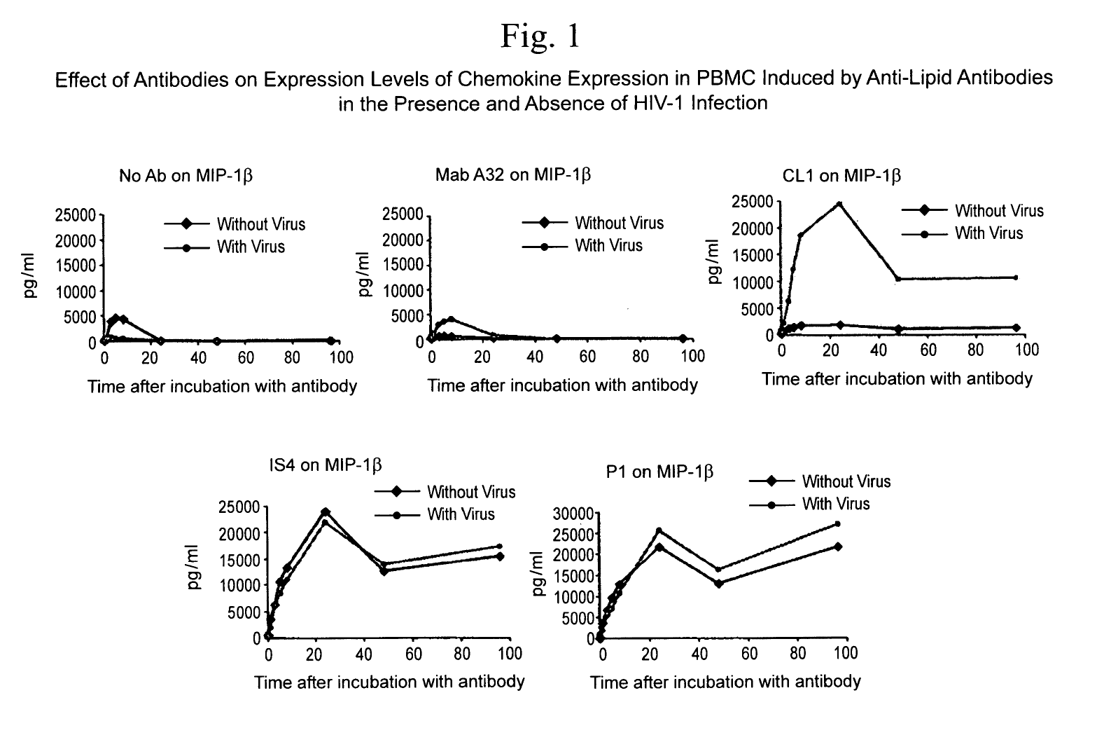

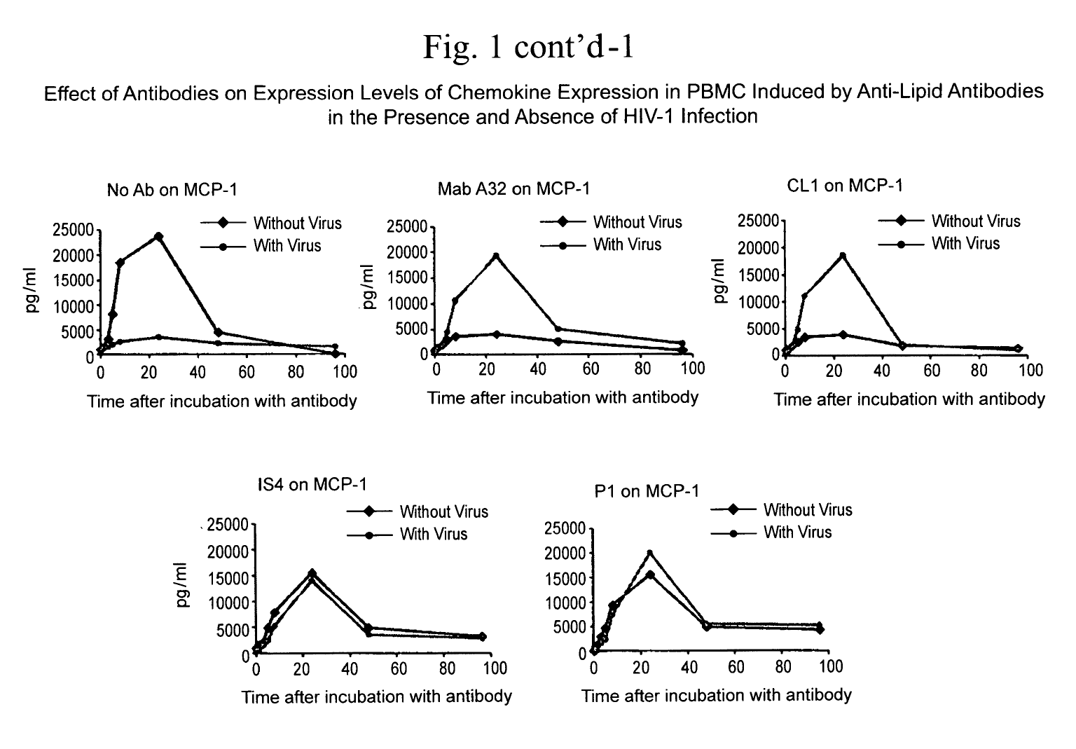

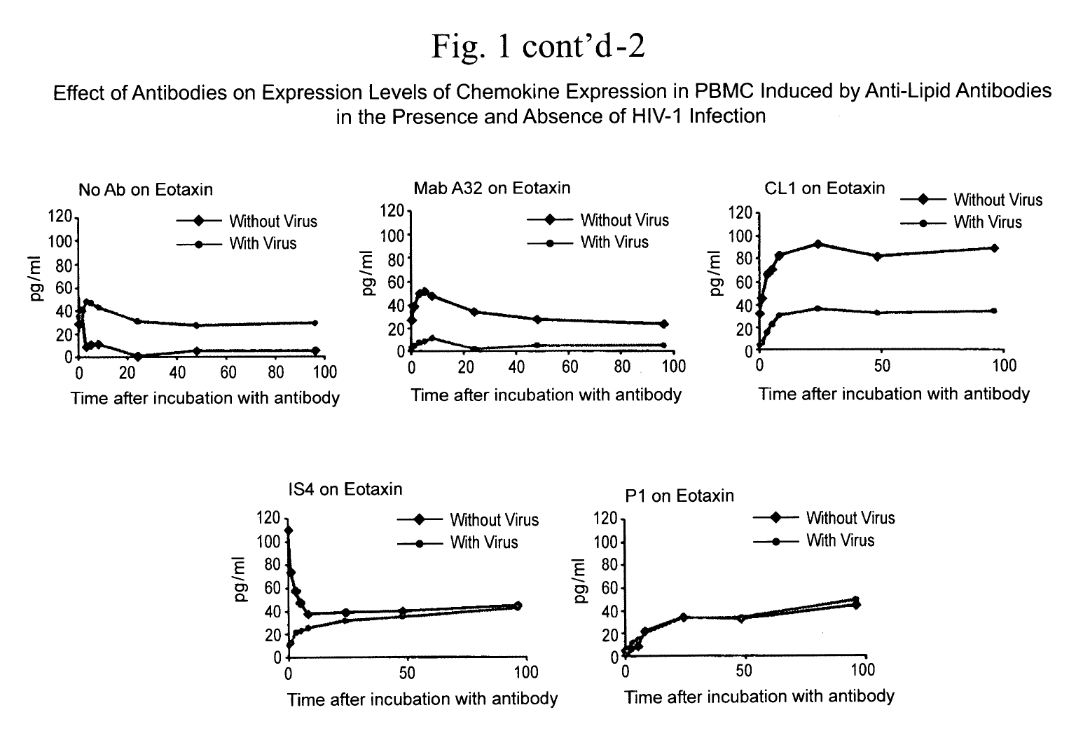

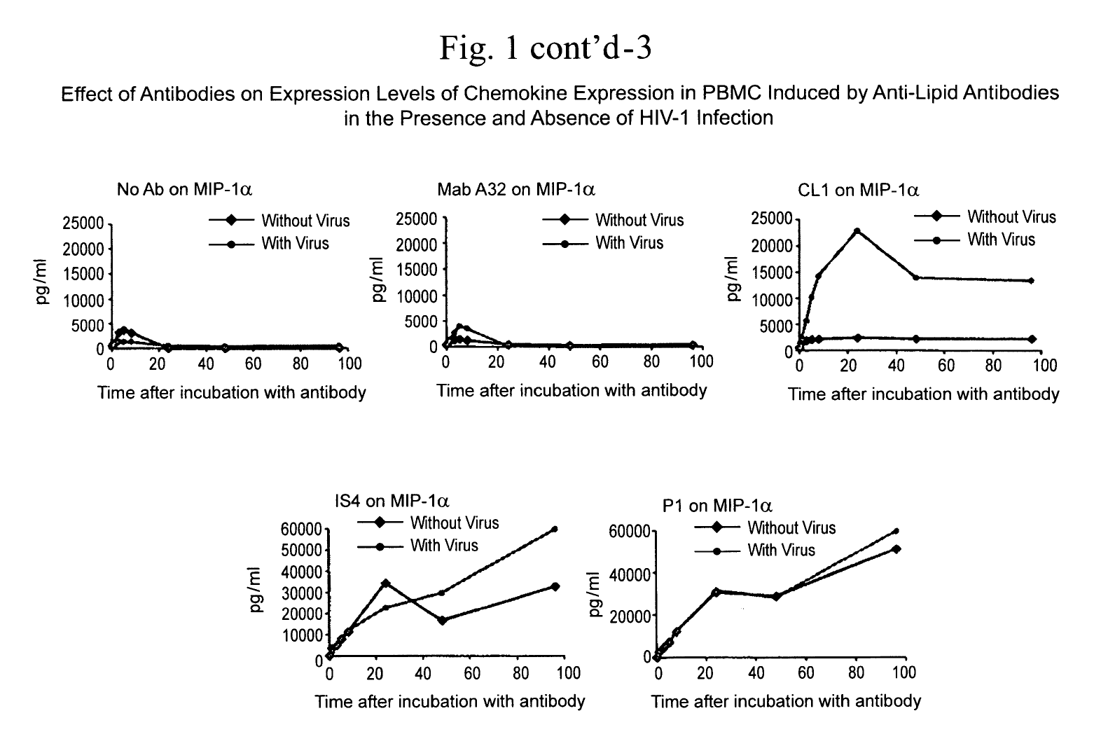

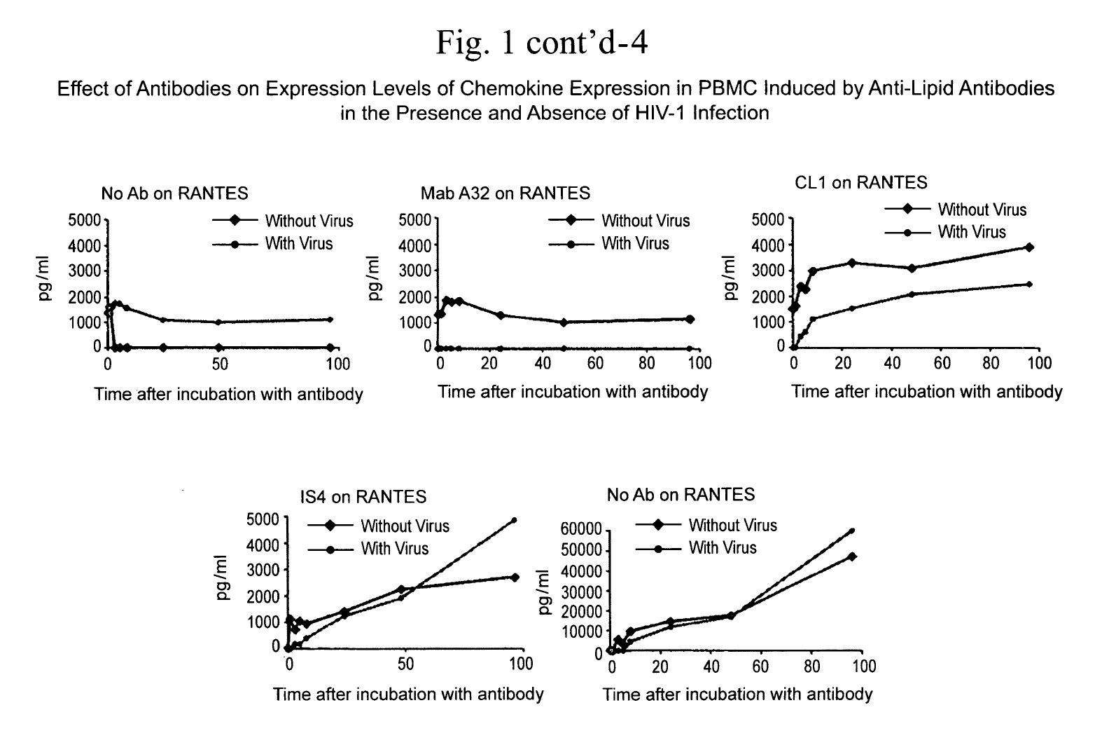

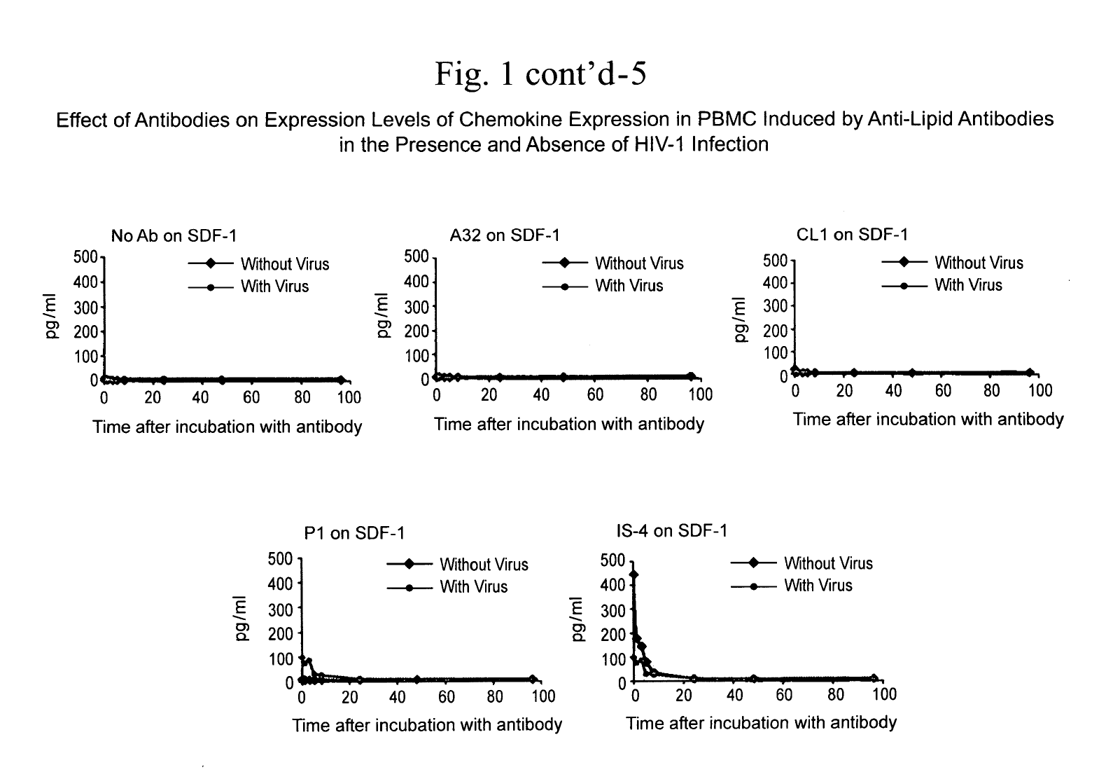

It has now been found that HIV-1 infectivity of isolated CD4+ T cells (though they are infectable with HIV-1), is not prevented by CL1, P1 or IS4 mAbs but, rather, that these antibodies can only prevent HIV-1 infection when peripheral blood monocytes are present (Table 4). A study of the effect of CL1, P1 and IS4 on production of chemokines that can prevent the infection of CCR5-utilizing, but not CXCR4-utilizing, HIV strains has demonstrated that:
1. P1, CL1 and IS4 mAbs induce production of the CCR5 ligands, RANTES (weakly), MIP-1α, and MIP-1β, but not the CXCR4 ligand SDF-1 (FIG. 1);
2. HIV-1 alone induces minimal amounts of these CCR5-binding chemokines in some subjects and robust amounts in others (FIG. 1); and
3. the combination of HIV-1 and one of the anti-lipid antibodies (any of P1, IS4 and CL1) leads to extraordinary production of CCR5 chemokines (FIG. 1).
   These observations have profound implications for the design of the present HIV-1 vaccine.

A CCR5 transmitted virus (WITO) and an CXCR4-utilizing transmitted virus (WEAU) engineered with a Luciferase reporter gene attached were used to infect PBMC. (See Table 5.) It was found that the anti-lipid antibodies all inhibited WITO infection of PBMC but did not inhibit WEAU HIV-1 infectivity of PBMC. Also used was EBV transformation of blood B cells from a subject with acute HIV infection (700-12-037) 132 days after HIV infection—mAb ACL4, an IgA dimer, was isolated from this subject (a heterohybridoma stable cell line of this B cell clone has been established). The resulting human mAb potently inhibits the transmitted R5 virus WITO thus demonstrating that the transmitted virus in subject 037 induced an anti-lipid antibody to be produced, thus, HIV-1 can boost or induce this type of antibody. In this case, the induction came too late to help the patient. This shows how to prime for this type of antibody. The ACL4 antibody isolation from patient 037 shows that HIV-1 can stimulate this type of antibody, thus making it possible for HIV to, in effect, boost this anti-lipid “self natural antibody” in an “HIV specific” manner. That is, by priming for a ACL4-type antibody using a vaccine before HIV-1 infection, makes it possible for HIV-1 to boost that same antibody immediately upon transmission. This approach makes it possible to inhibit HIV within hours (e.g., within 48 hours) of infection and thus to extinguish HIV-1.

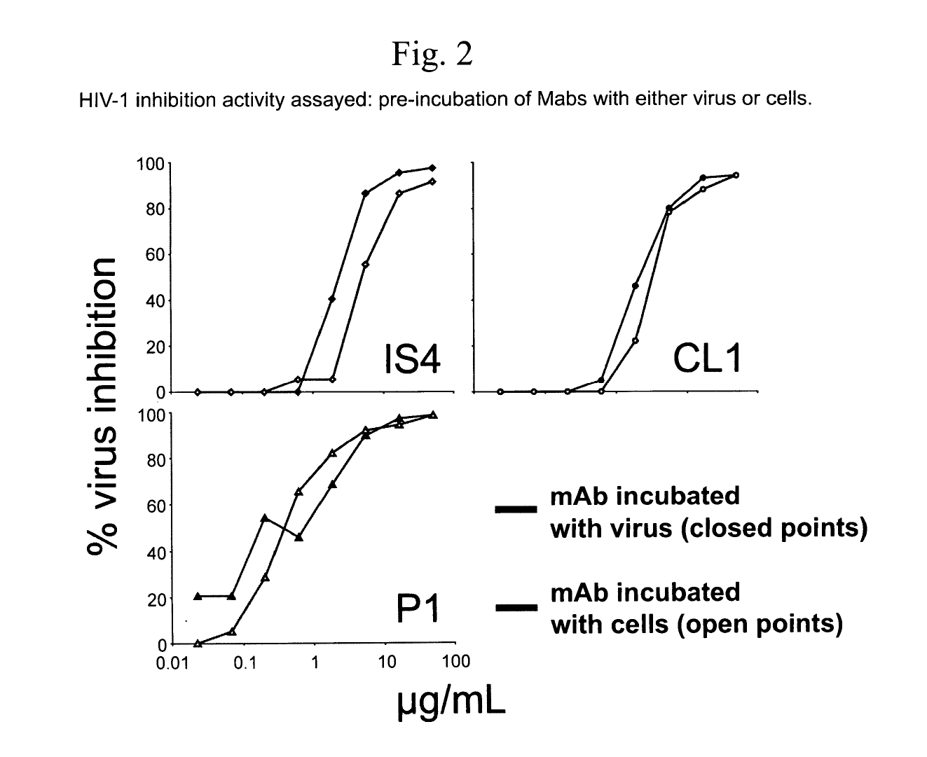

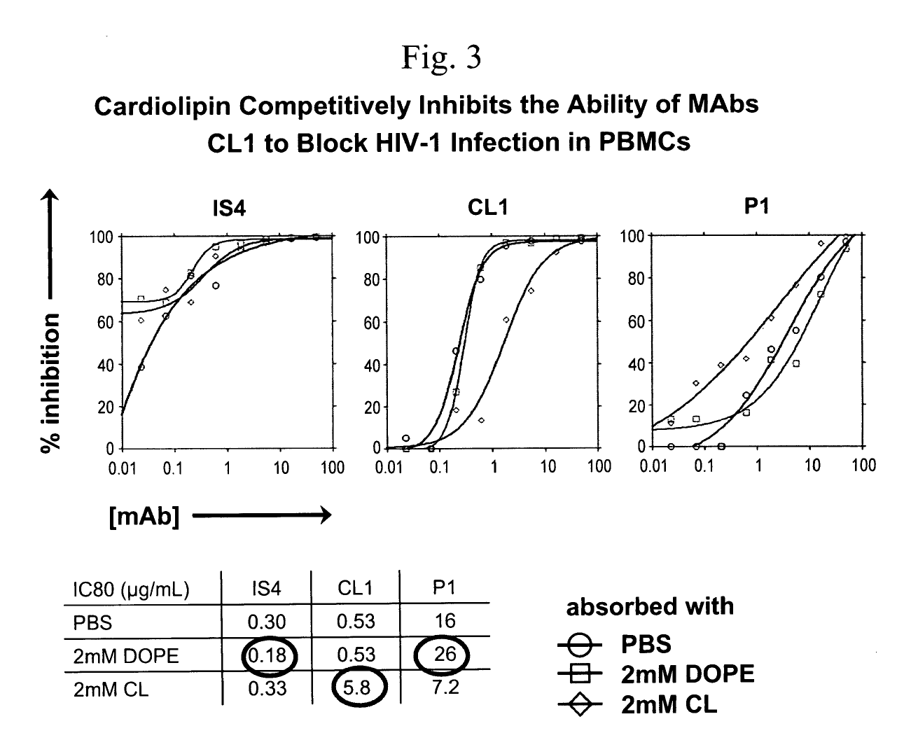

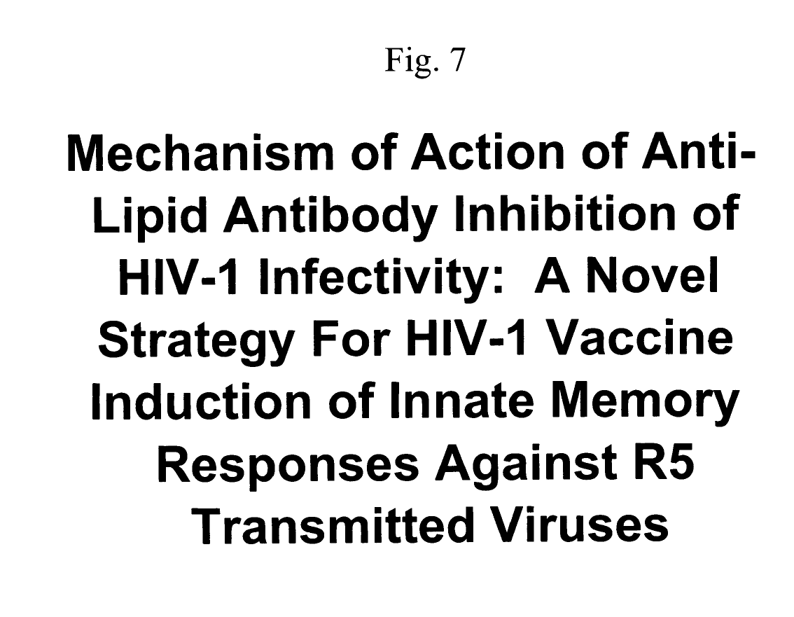

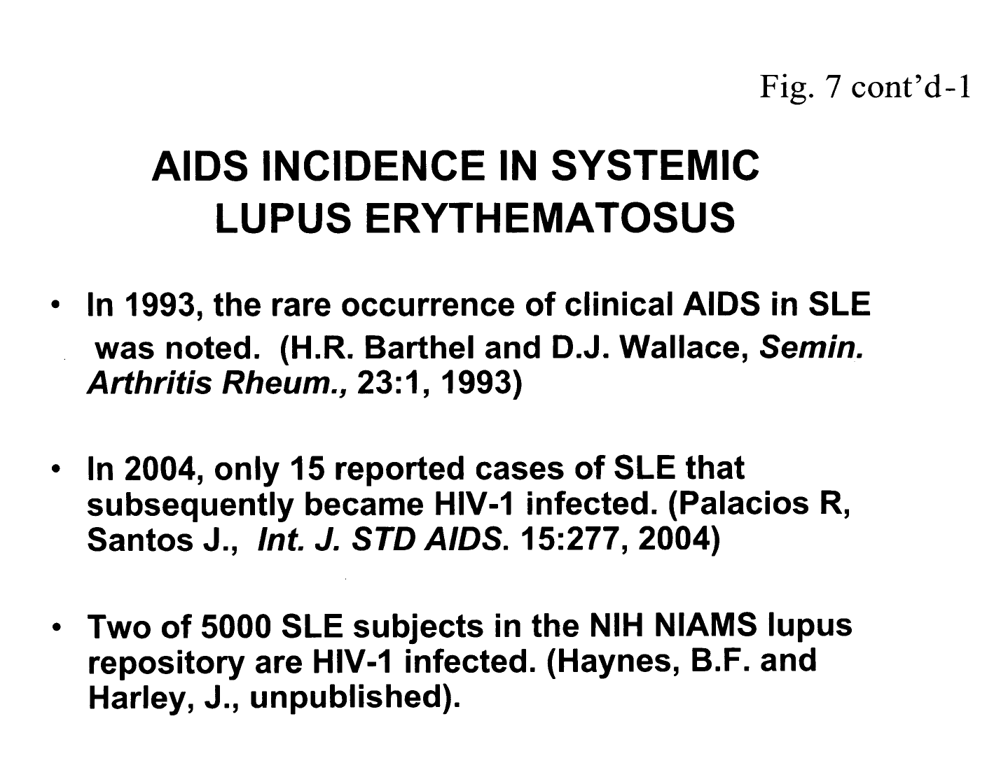

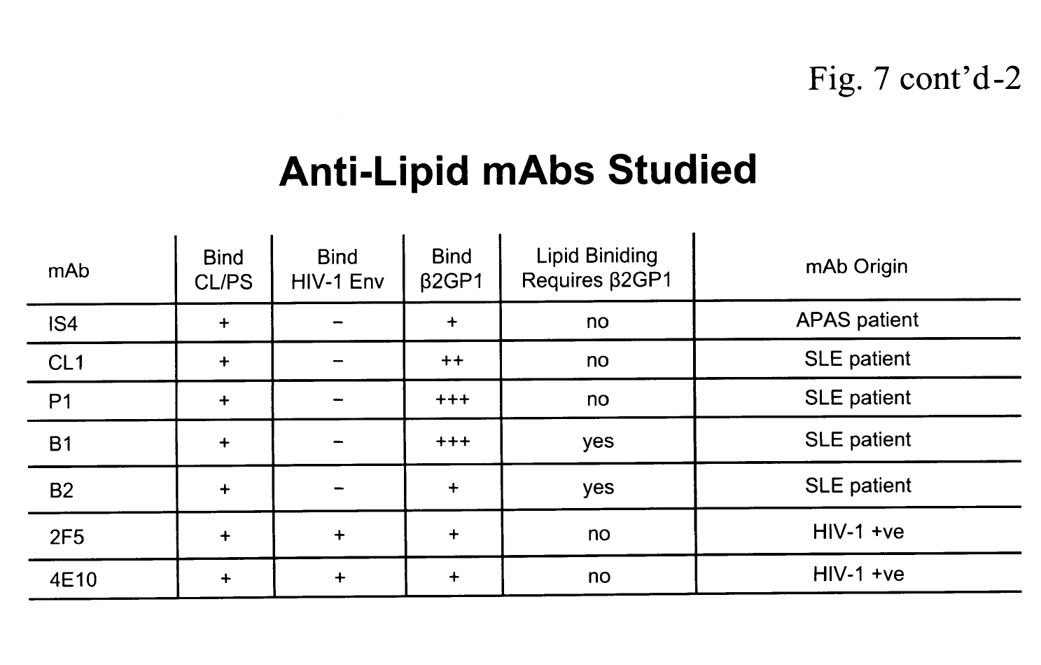

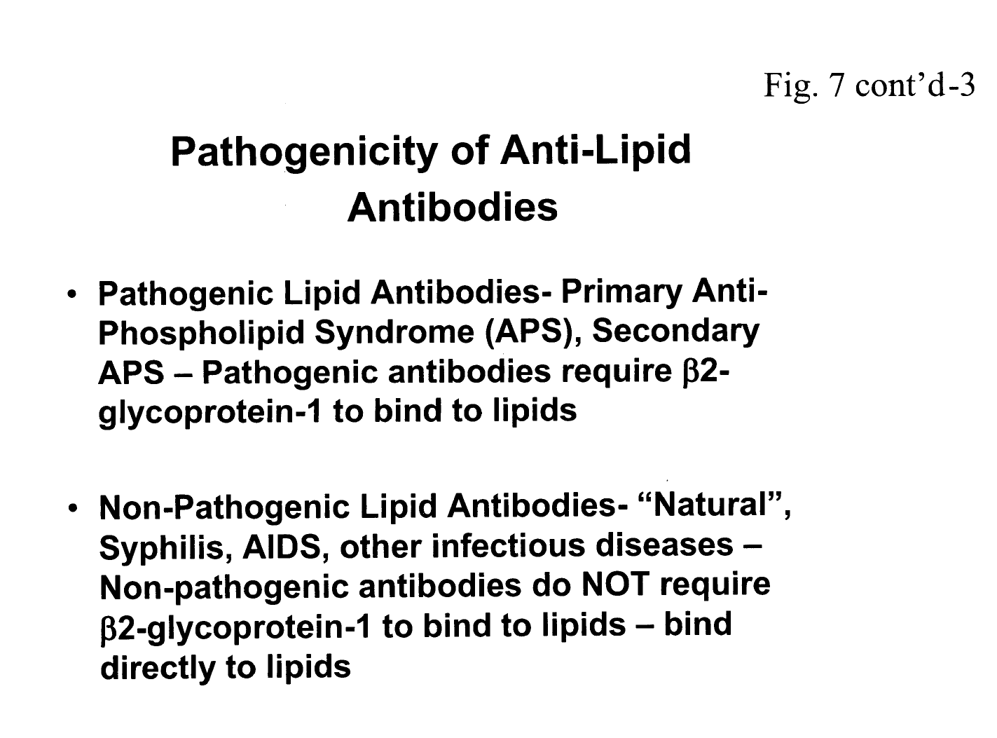

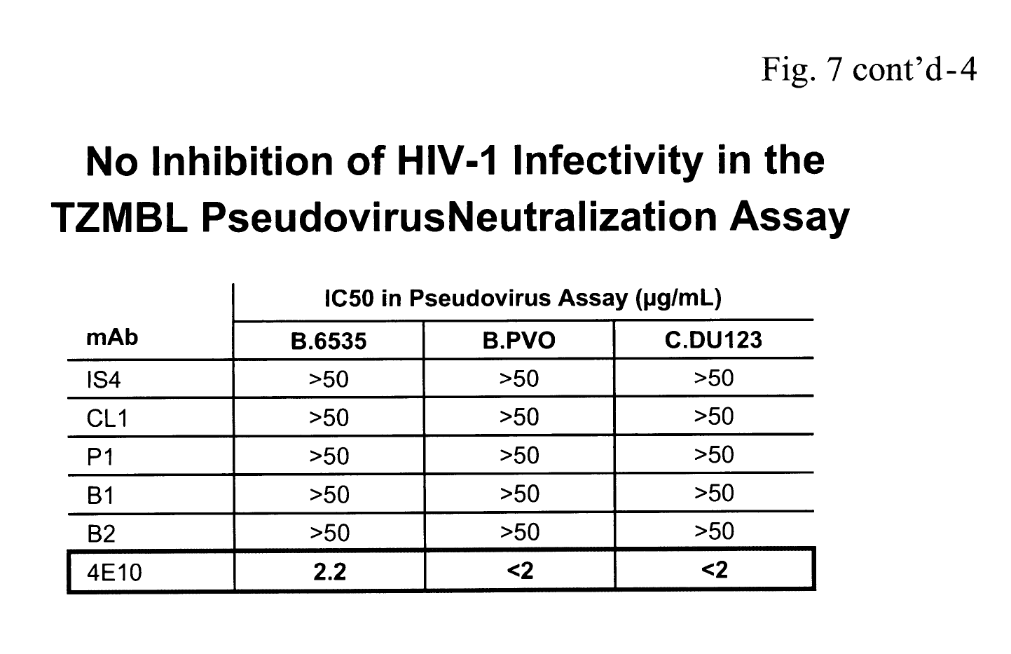

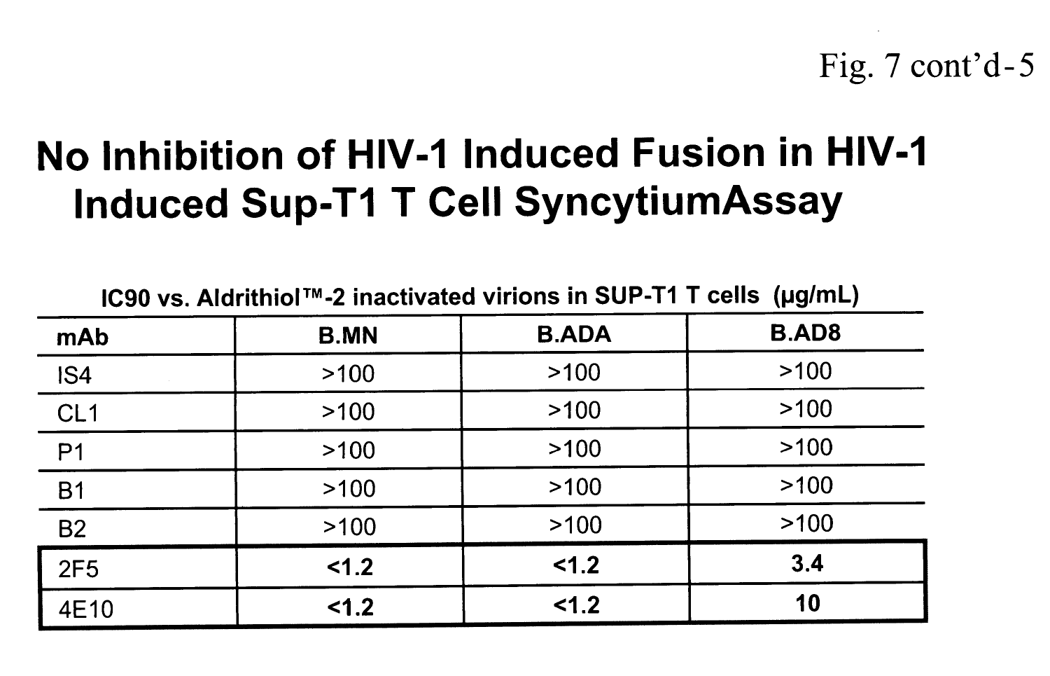

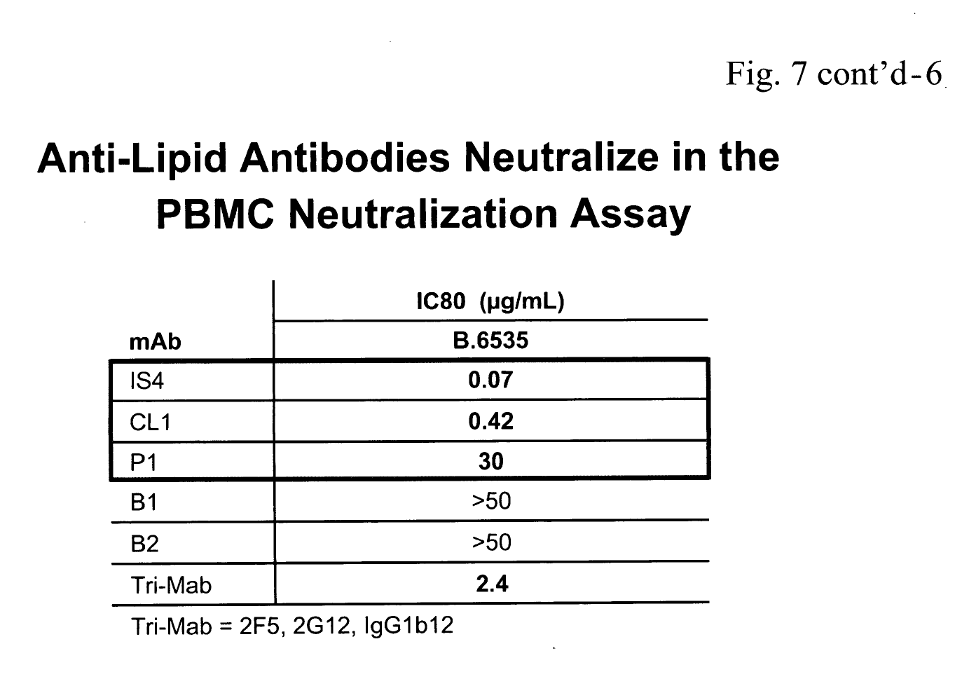

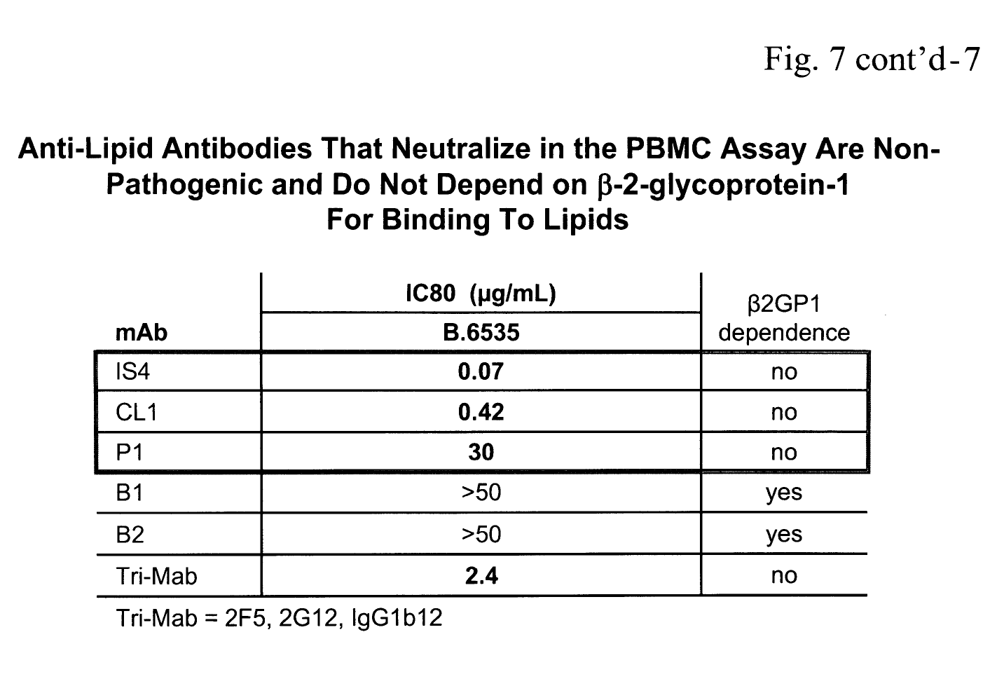

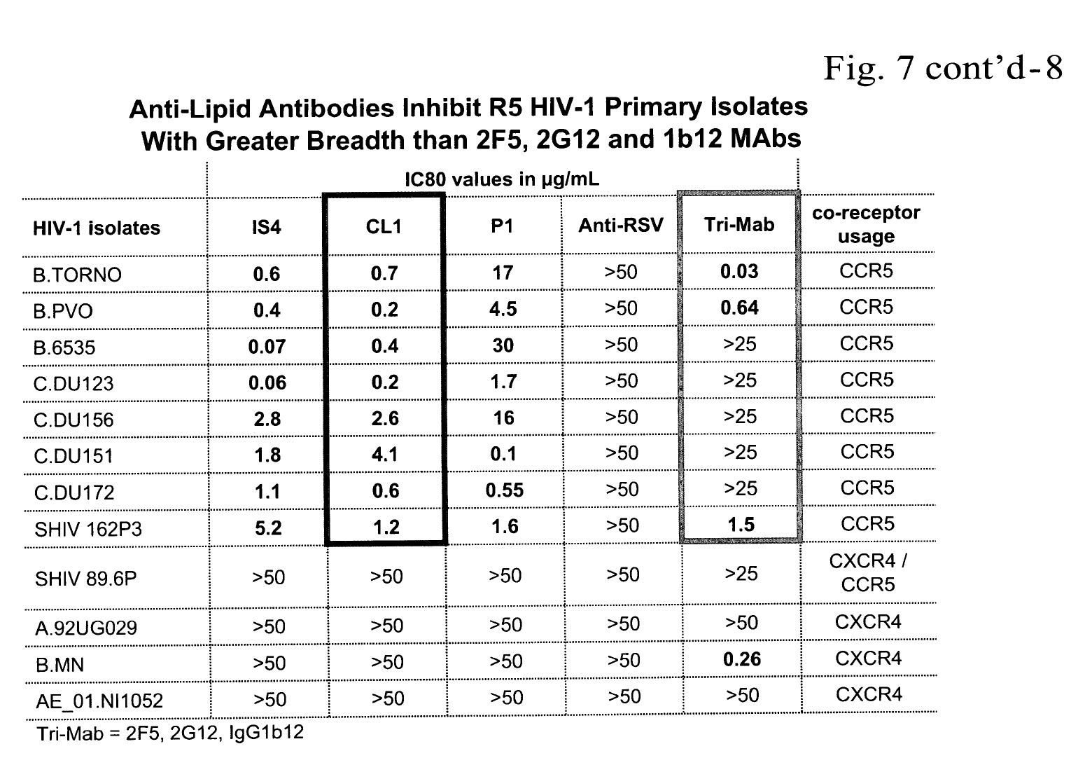

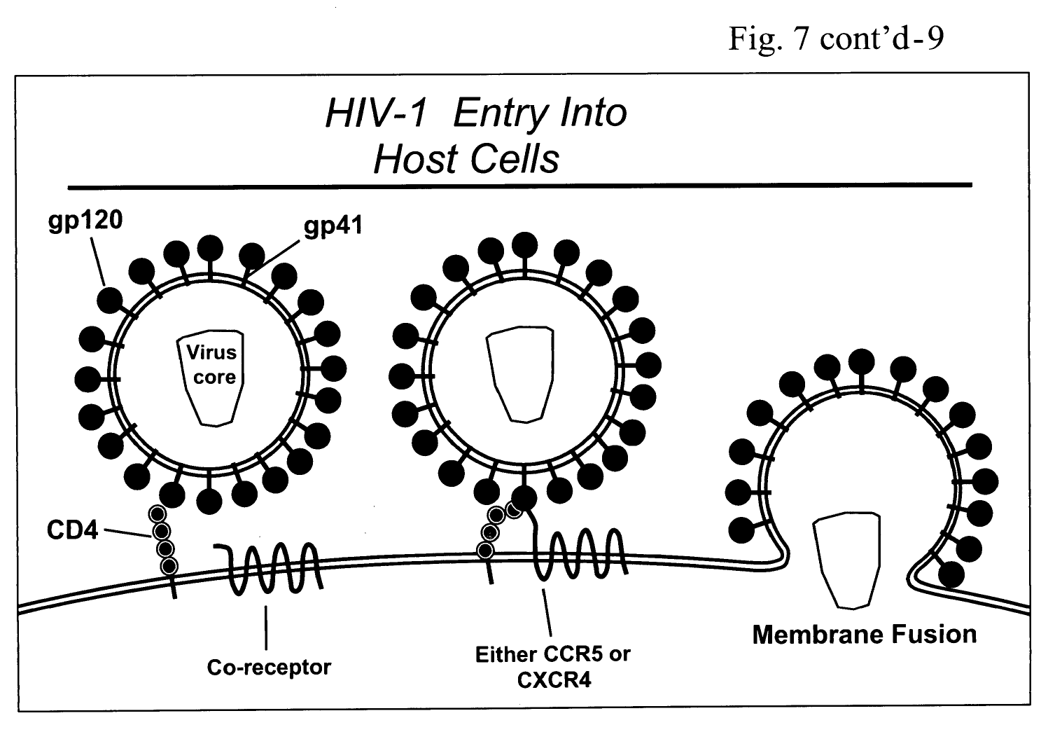

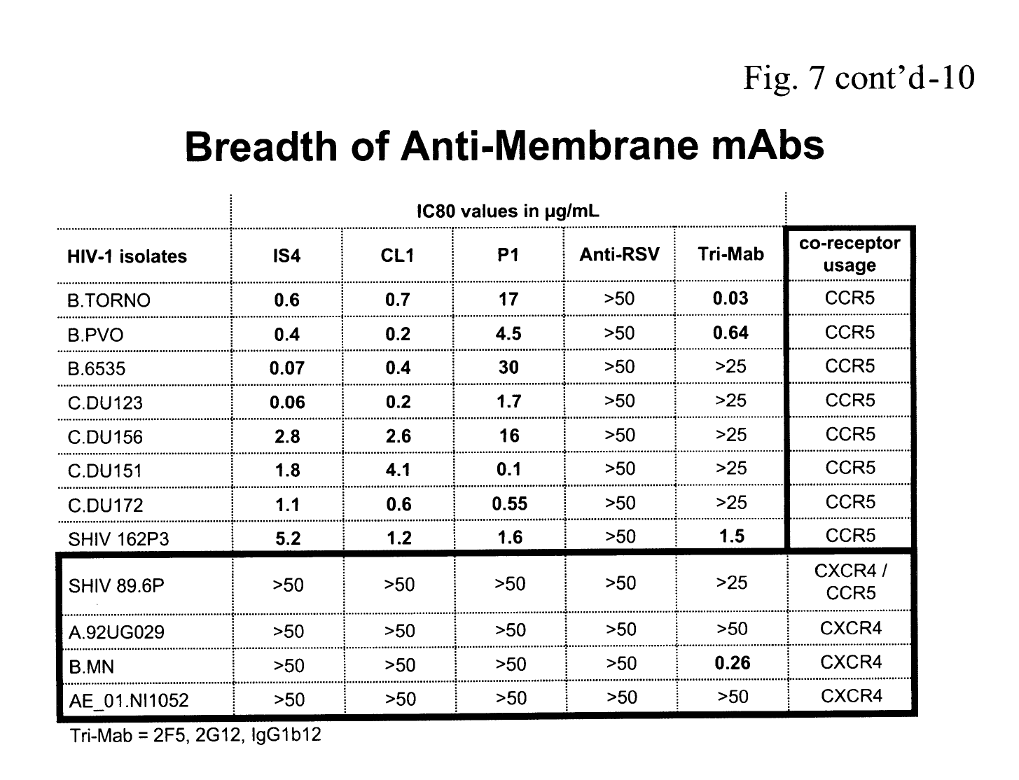

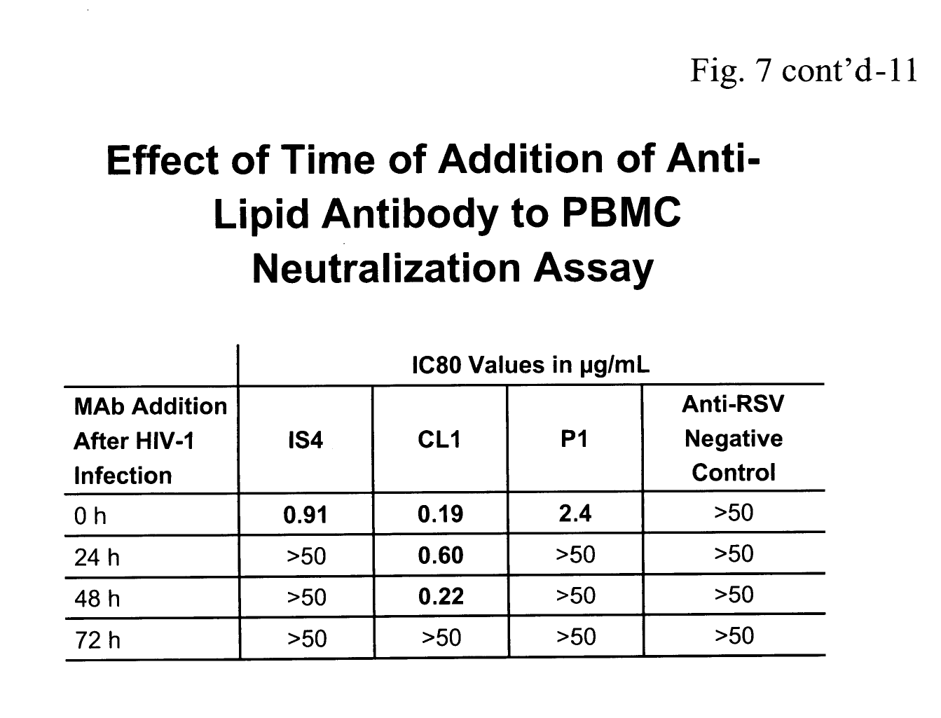

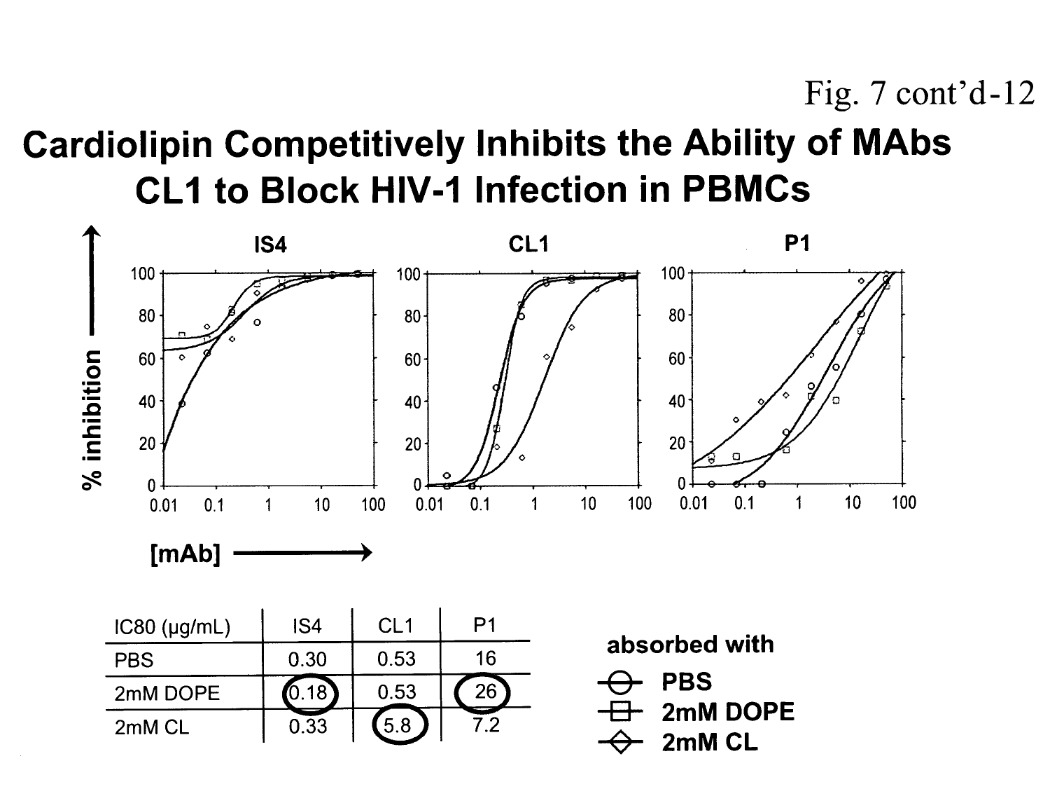

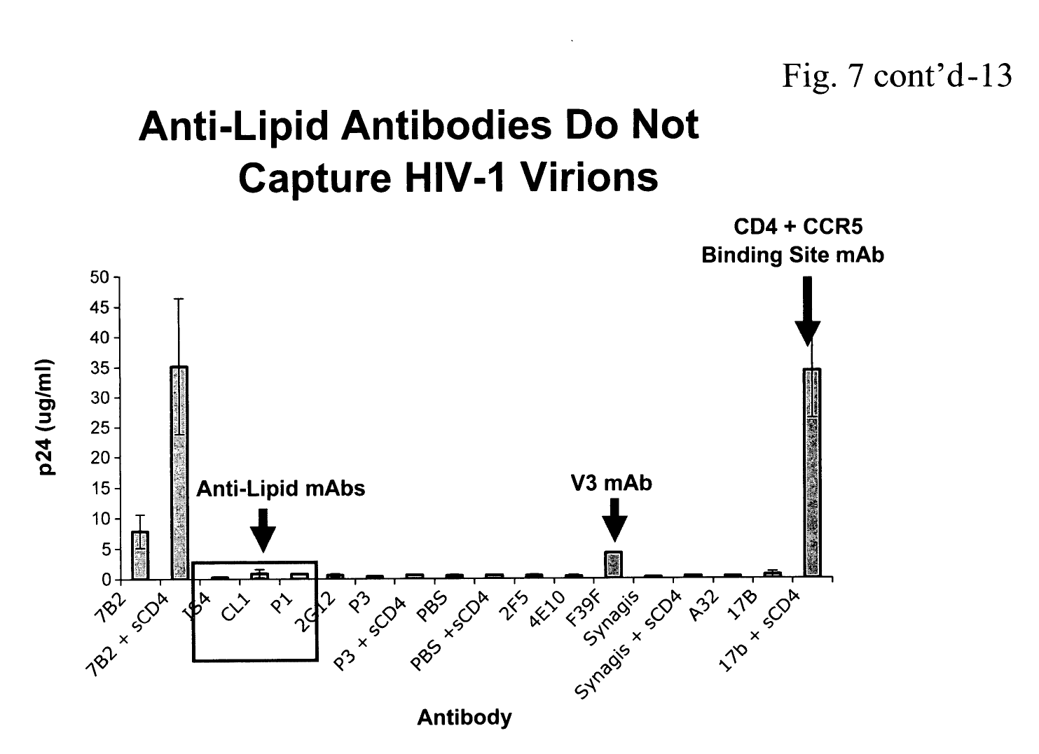

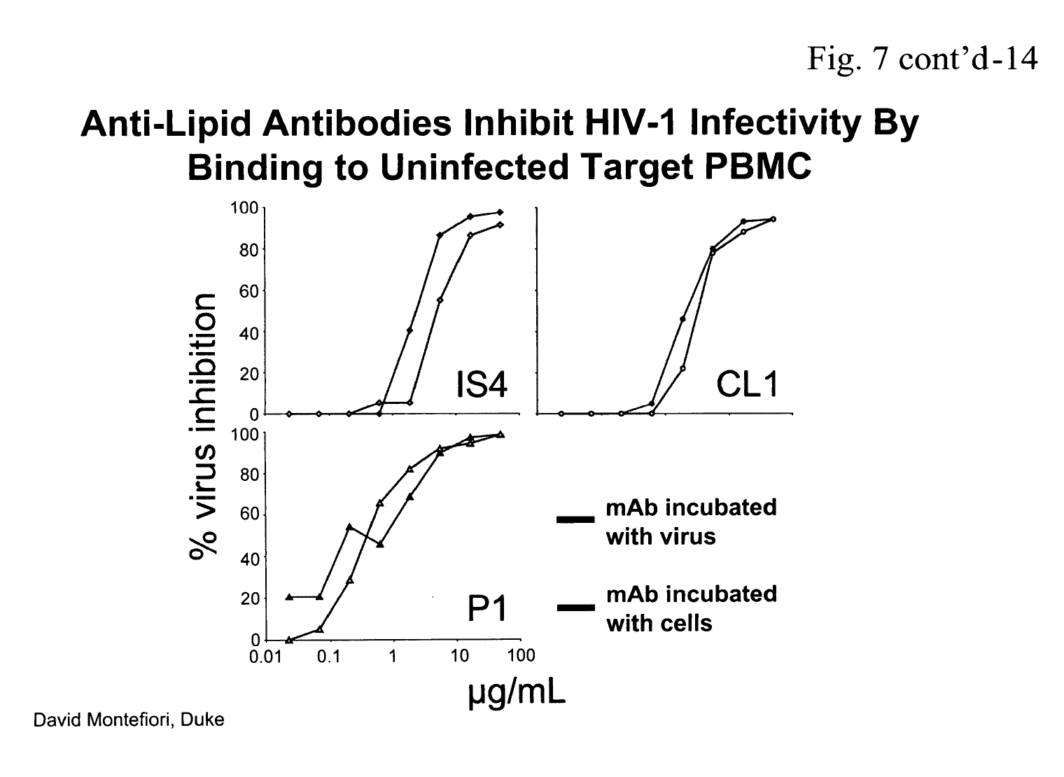

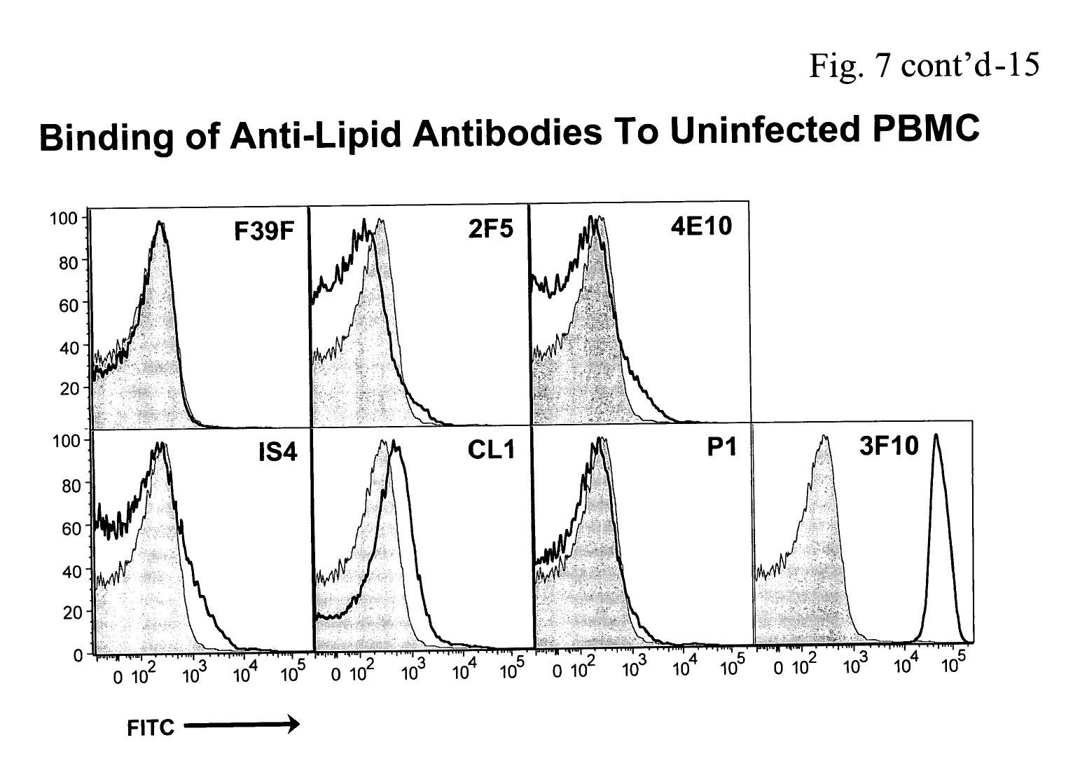

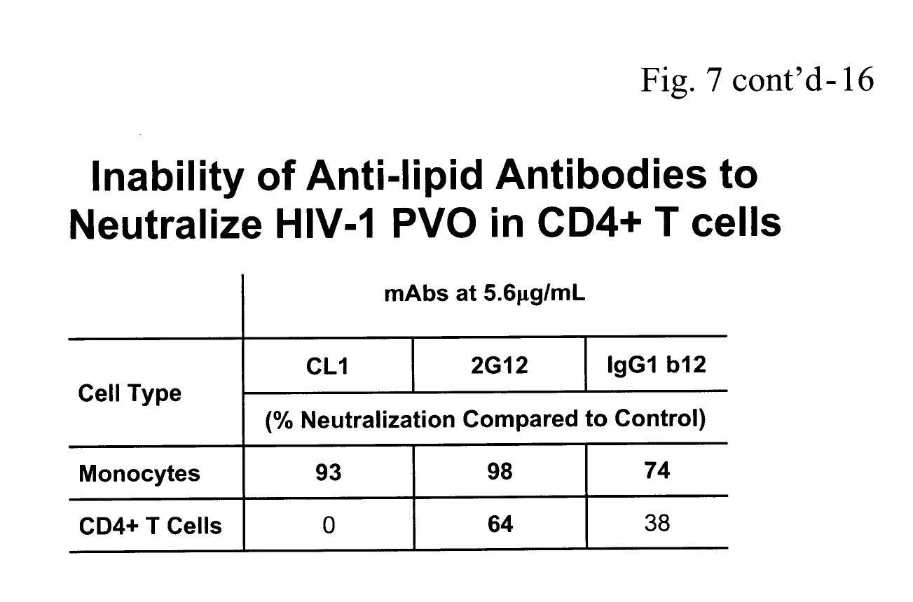

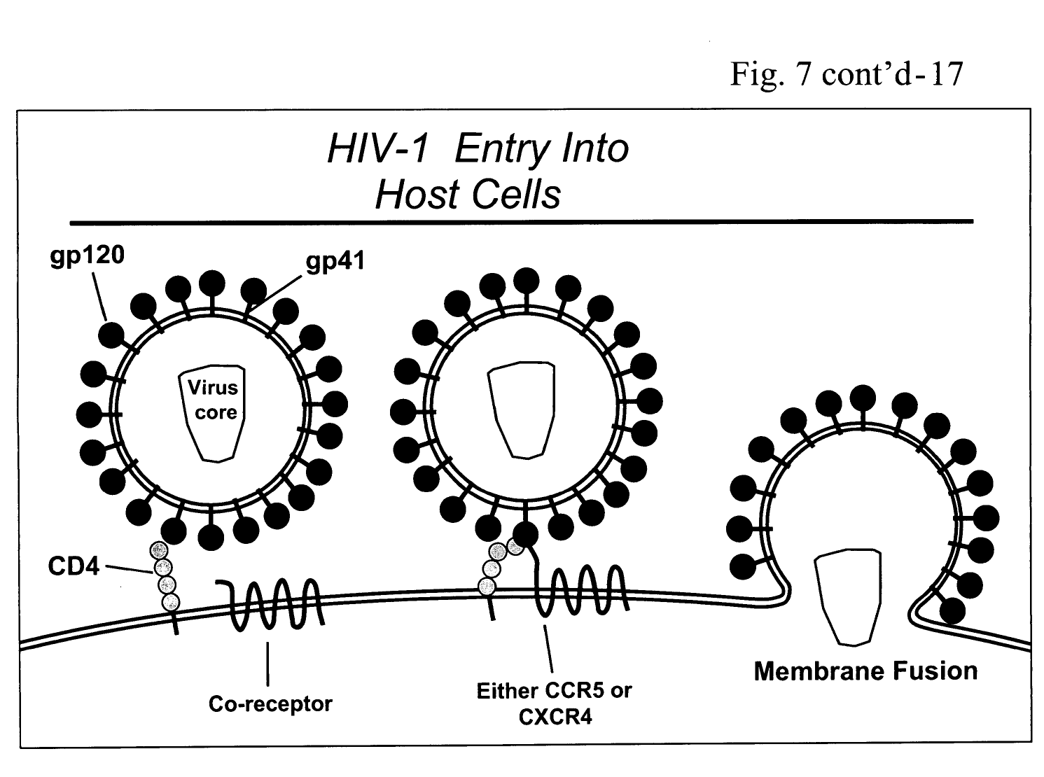

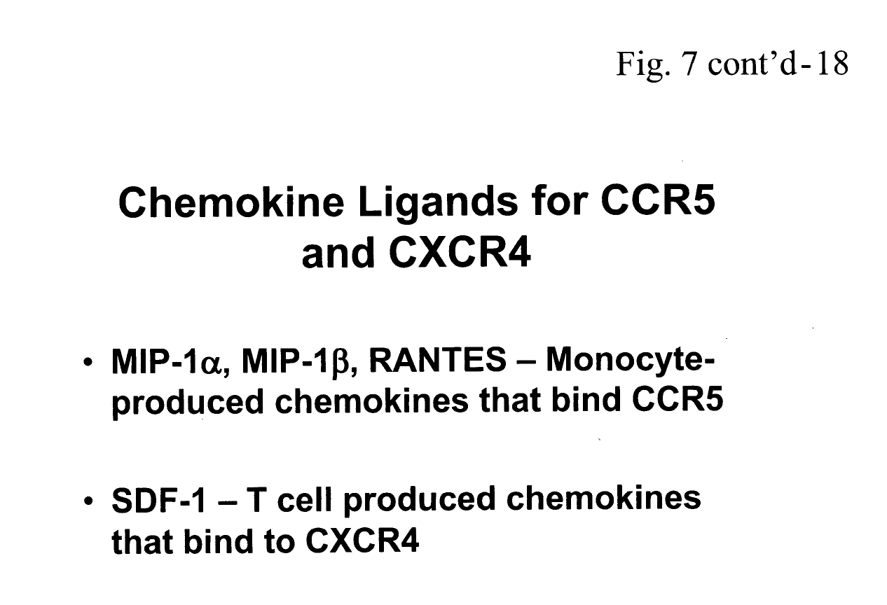

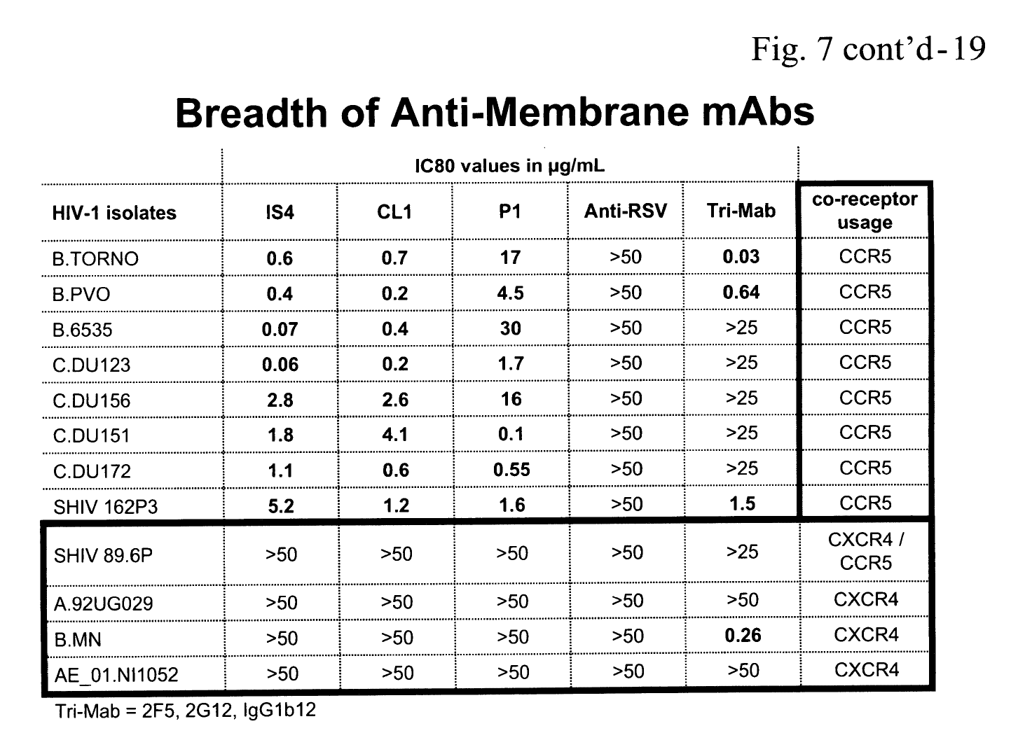

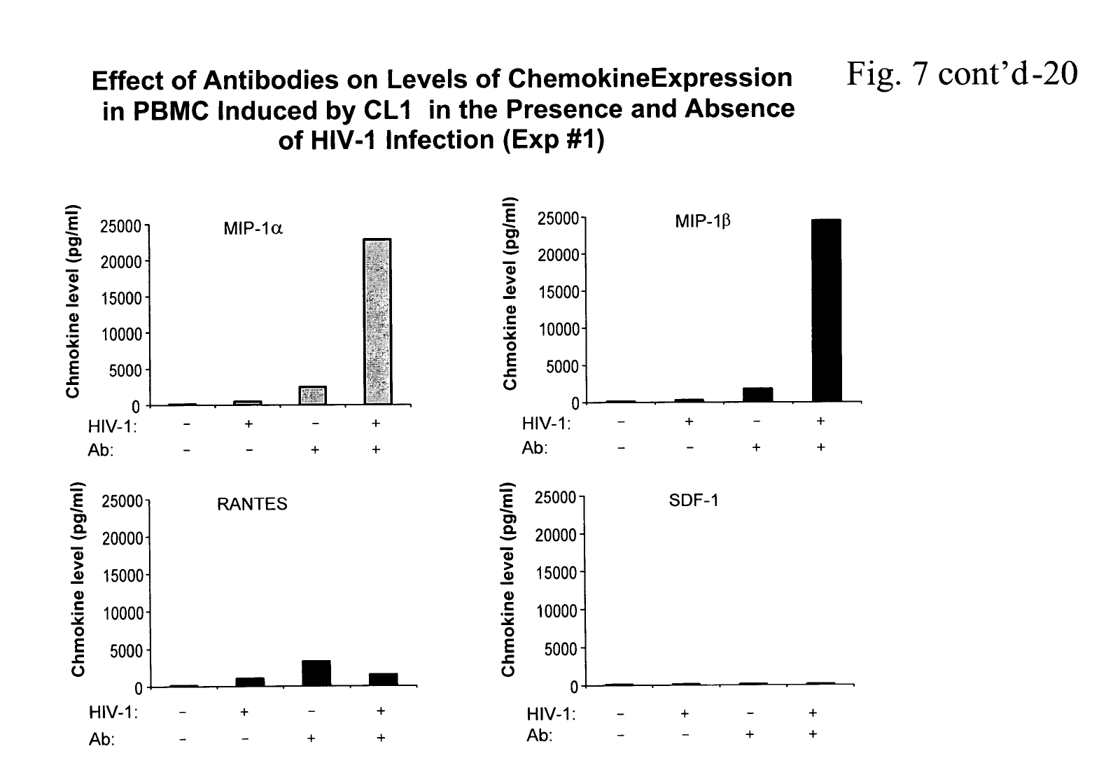

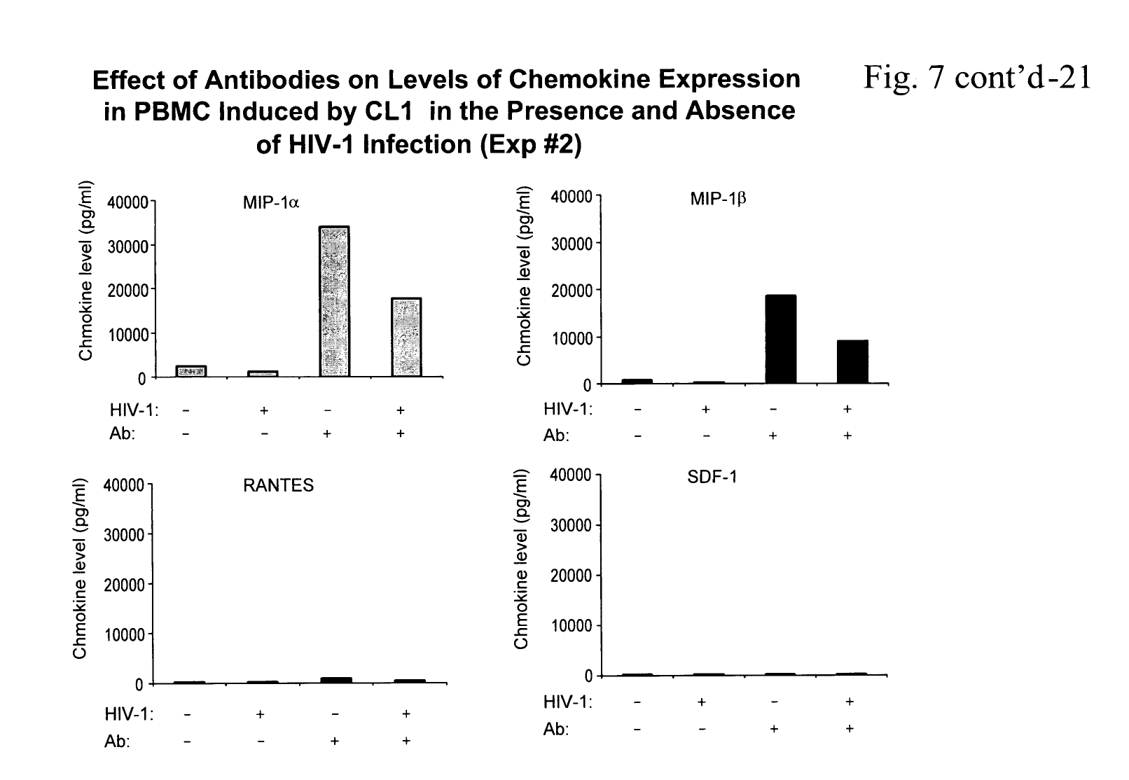

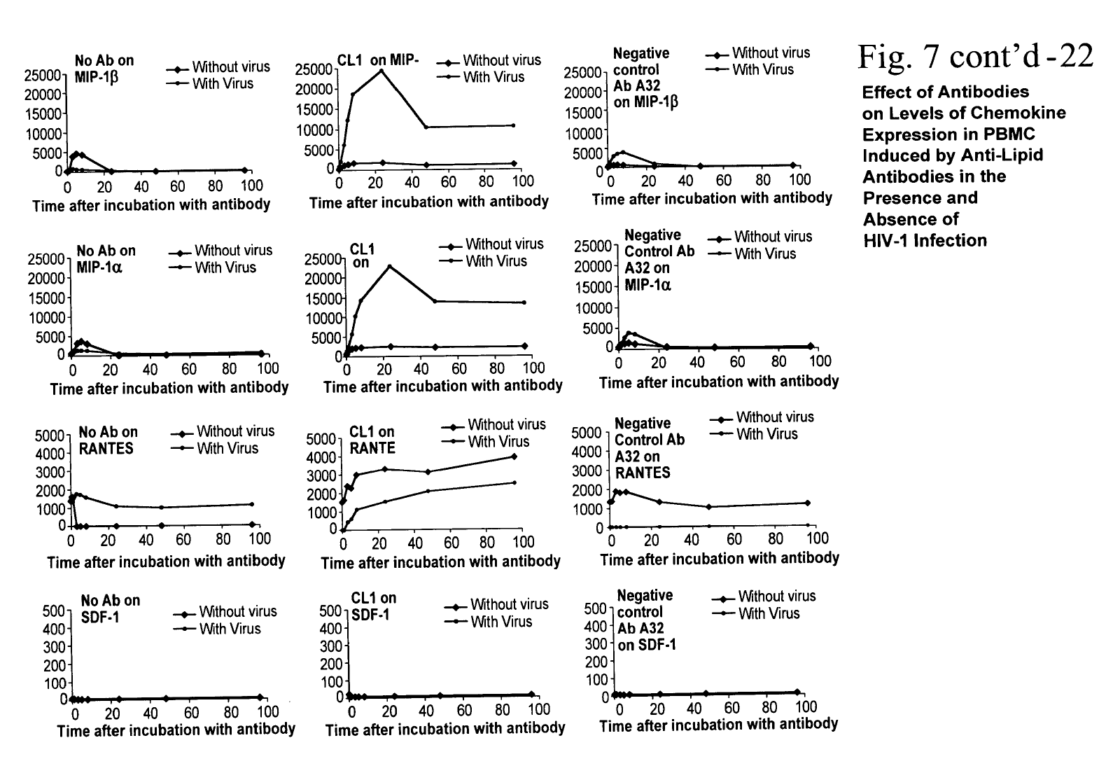

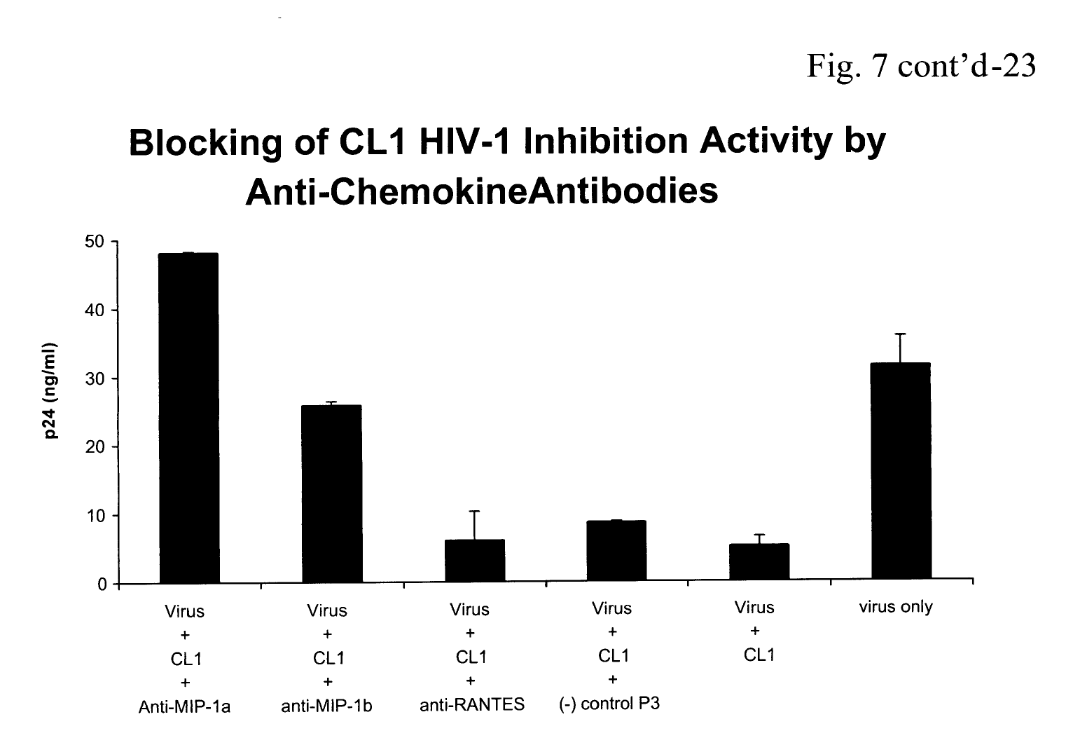

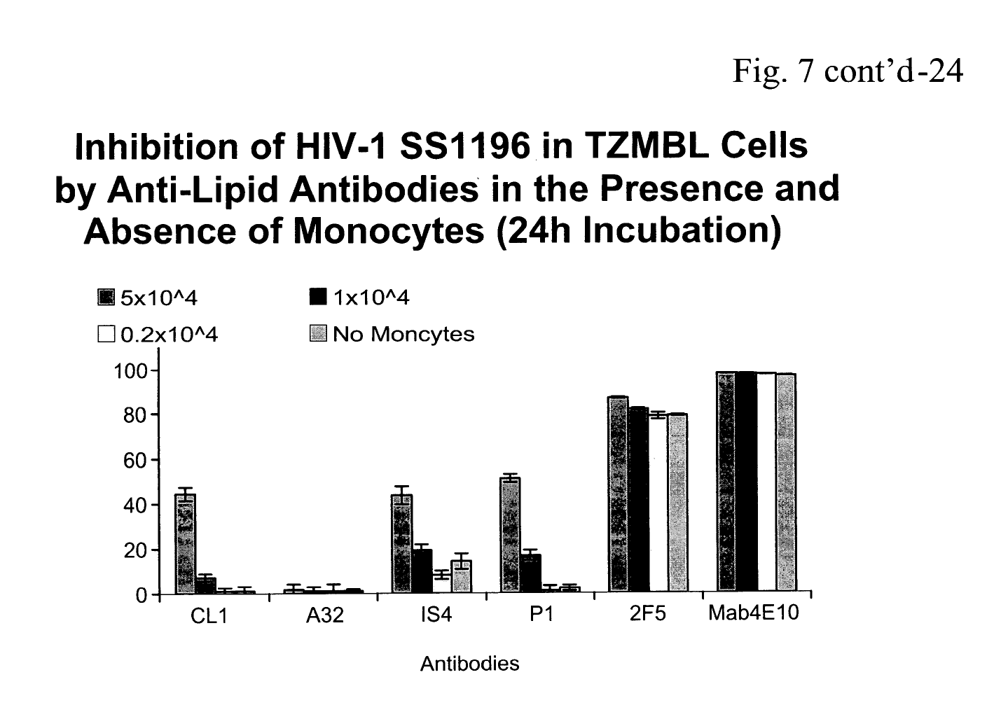

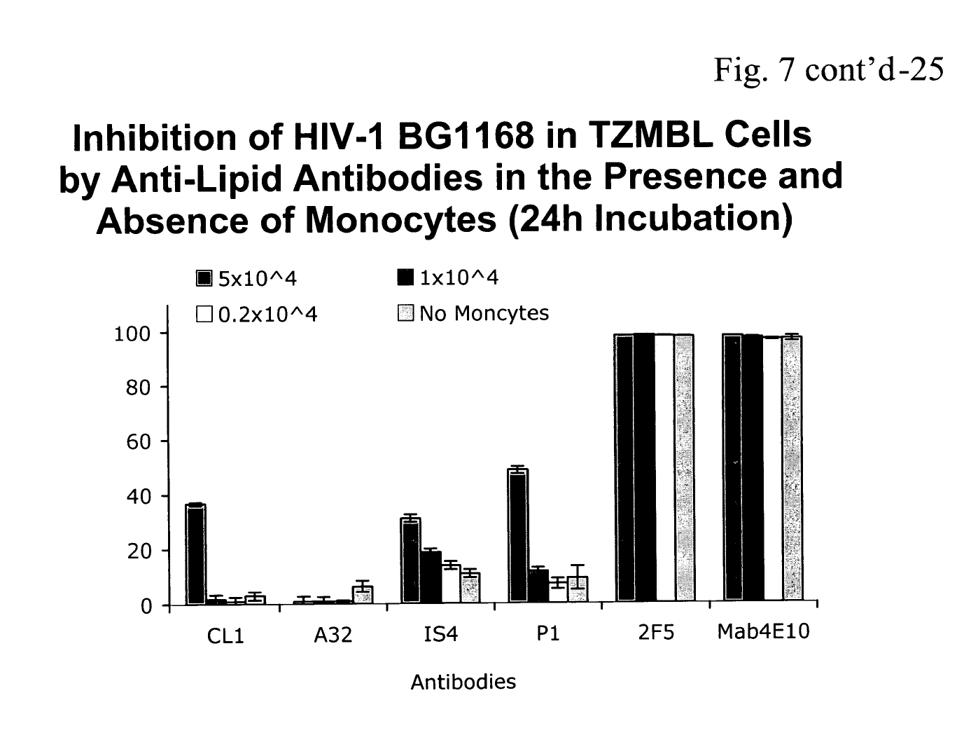

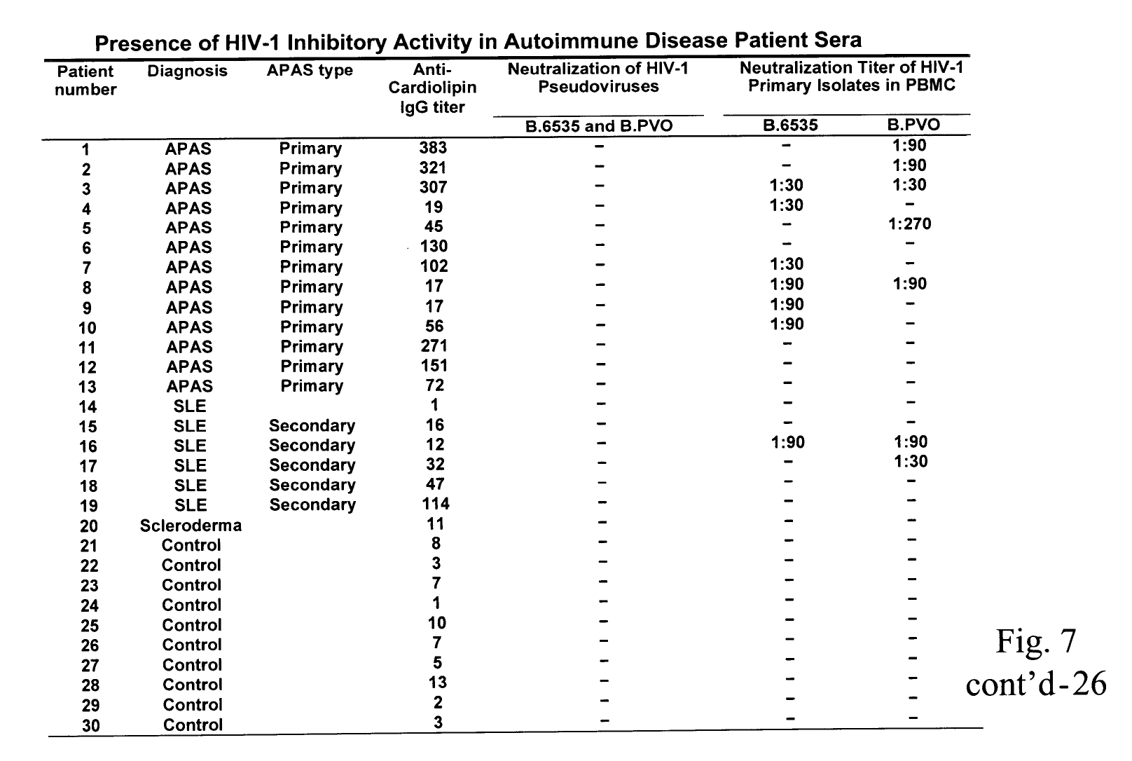

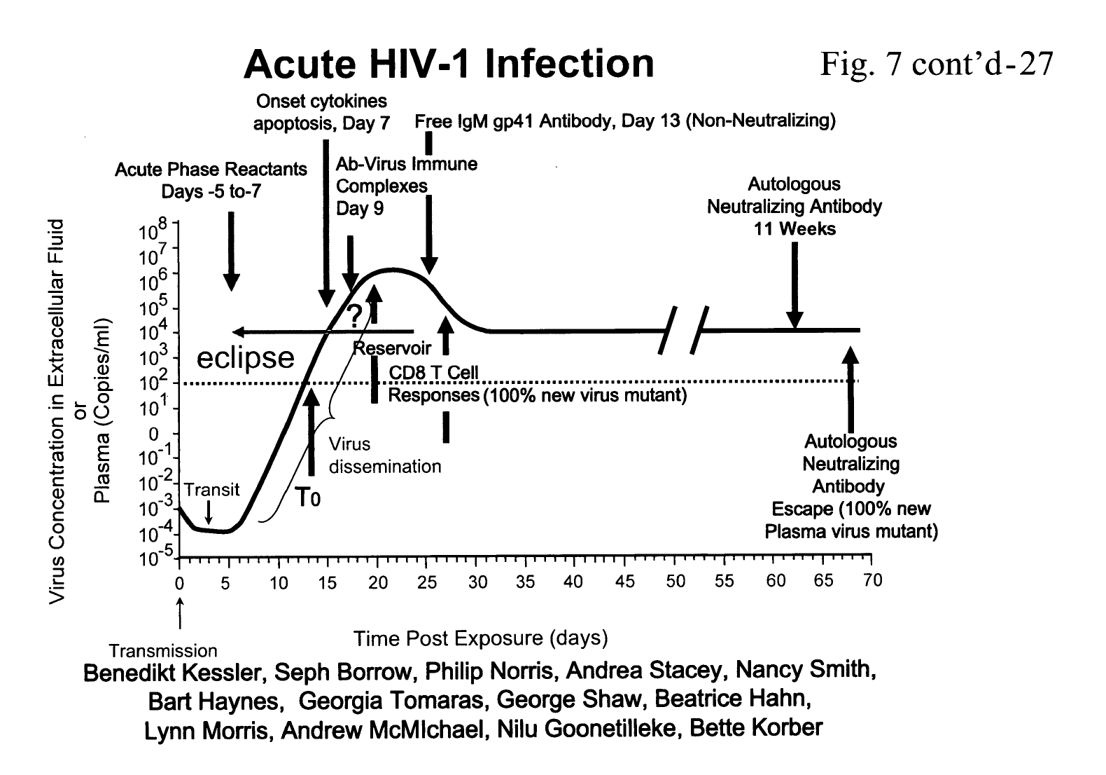

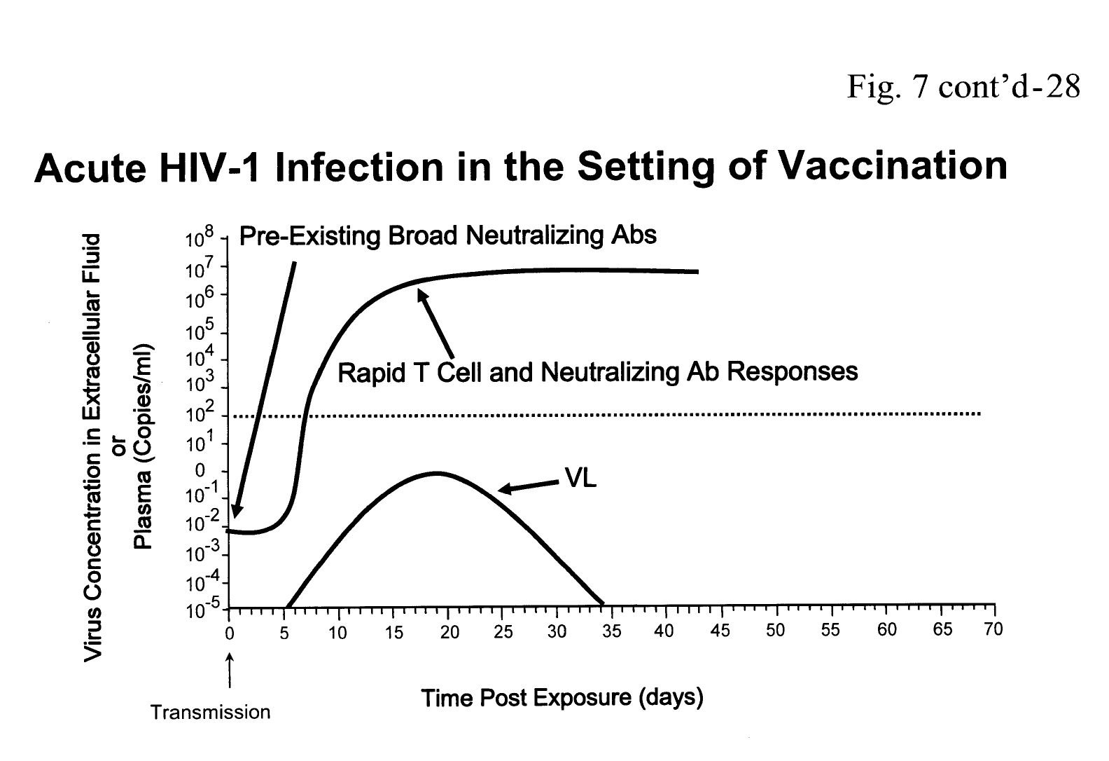

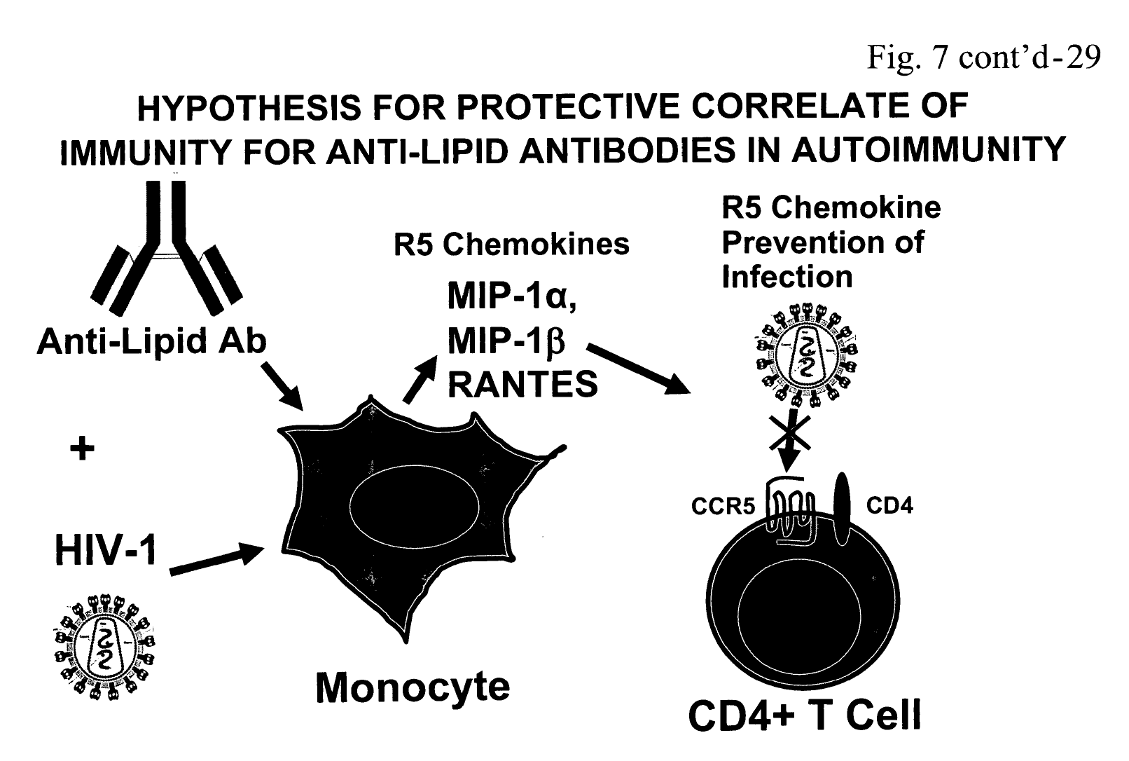

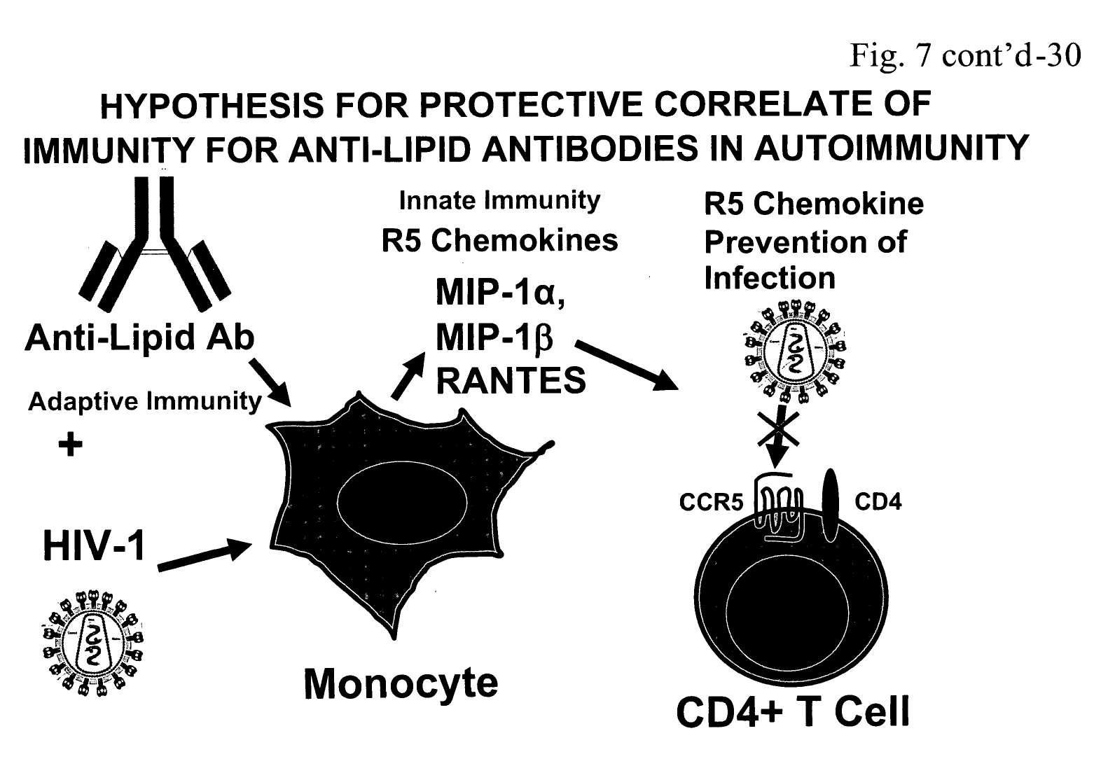

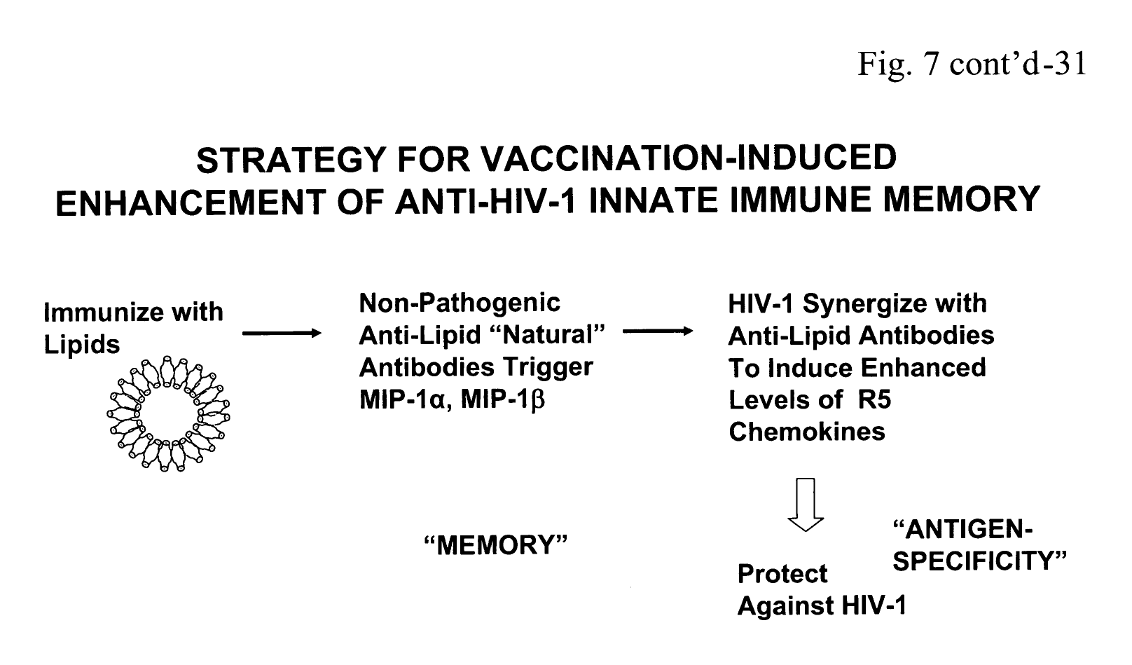

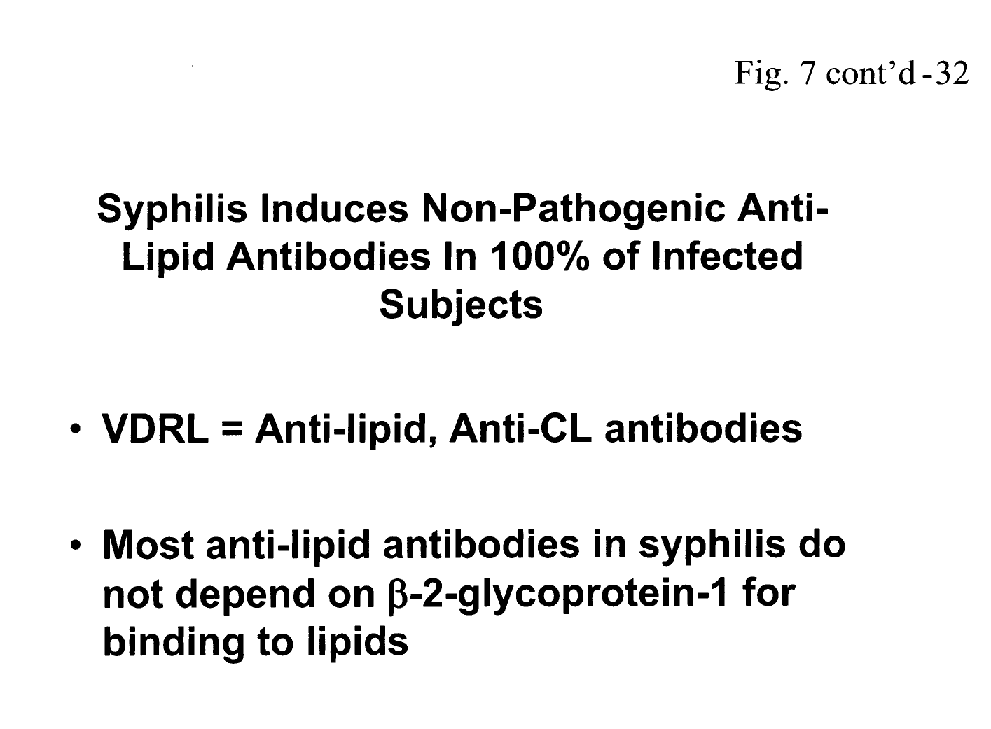

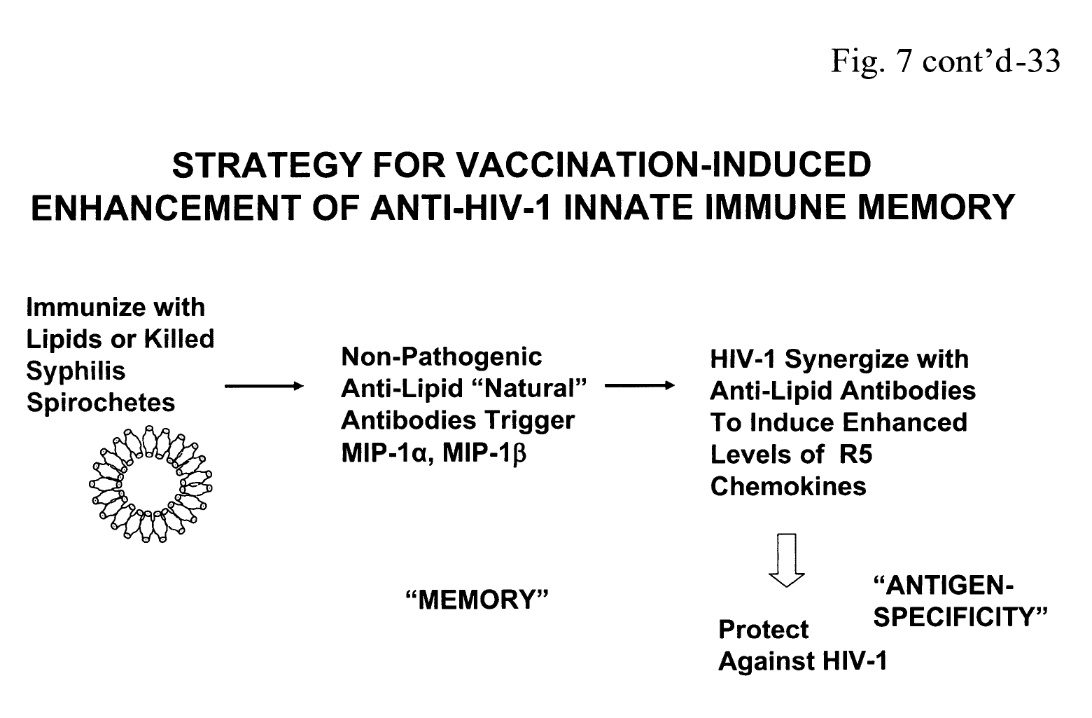

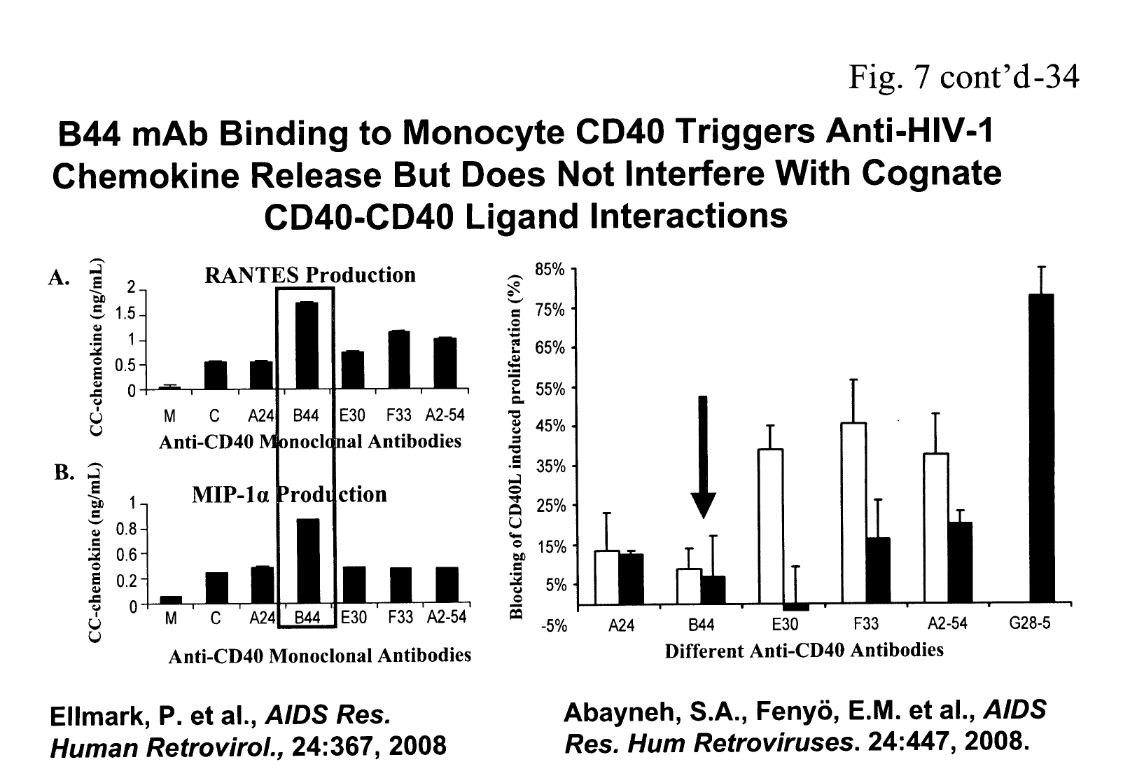

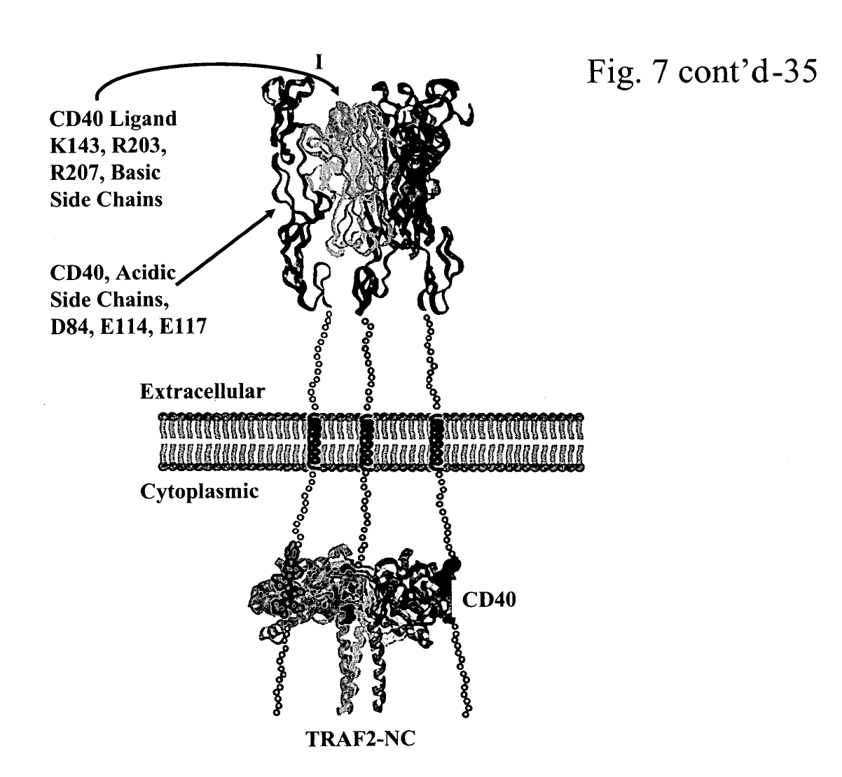

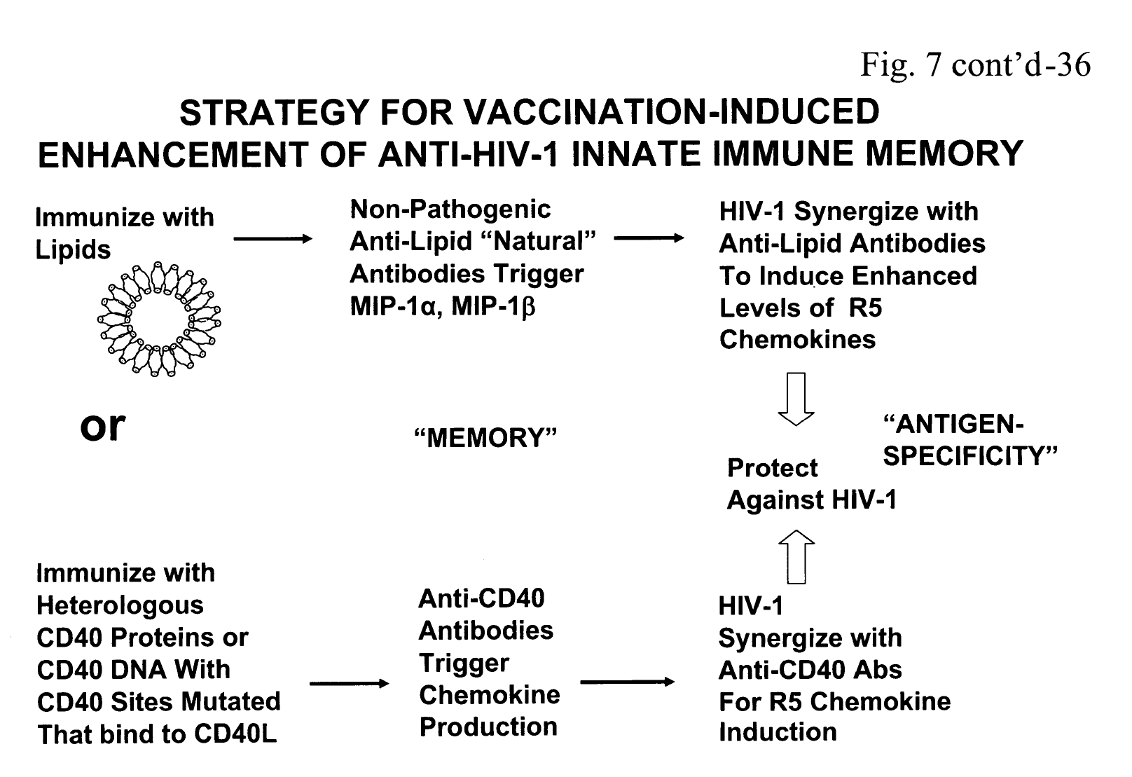

It was also noted previously that P1, CL1 and IS4 mAbs bind to host PBMC and inhibit by binding to host cells rather than to virions (FIG. 2). Moreover, P1, CL1 and IS4 mabs bind to PBMC cells in a pattern suggestive of lipid rafts. It has also been shown previously that the virus-inhibiting activity of P1, IS4 and CL1 can be inhibited by lipids such as cardiolipin (FIGS. 3 and 7)—that is, these antibodies can bind cardiolipin in vitro as well as PS and PE (Zhu et al, British Journal of Haematology 105:102-109 (1999), Lin et al, Arthritis & Rheumatism 56:1638-1647 (2007)). However, cardiolipin is not in the outer cell membrane but rather is in the mitochondrial membrane, thus PS and PE are the targets. Moreover, PS and PE are expressed on the cell surface of apoptotic cells but less so on the surface of viable cells (it has been appreciated recently that smaller amounts of PS and PE are present on the surface of viable cells (Balasubramanian et al, J. Biol. Chem. 282:18357-18364 (2007))).

### Example 2

Sequence alignment of wild-type CD40 of human (hCD40), mouse (mCD40) and rhesus monkey (RhCD40) and rhesus monkey CD40 as well as mouse CD40 and rhesus monkey mutant CD40 is shown in FIG. 5. Amino acids of human CD40 interfacing with CD40 ligand are bolded and underlined. A mutant mCD40, mCD40mutEK, was designed, in which amino acid (K114) at the corresponding interface of mouse CD40 was mutated to E as the same for human CD40 to avoid inducing antibodies that might interfere with the interaction of CD40 and CD40 Ligand. In the spanning region (indicated with a box) of the interface of CD40 with CD40 Ligand (from amino acid position 83 to 117), there are 2 amino acids (amino acids at positions 109 and 112) in the region that are different between rhesus monkey and human CD40 proteins, and there are substantial differences in sequences between mouse and human CD40. To minimize the potential for induction of antibodies that might interfere with the interaction of CD40 and CD40 Ligand, due to the amino acid differences in this spanning region, another mouse CD40 mutant (mCD40d81-114) and a rhesus CD40 mutant (RhCD40d109/112) were designed with their amino acid sequences in the interface spanning region mutated from the wild-type to the sequences as human CD40.

### Example 3

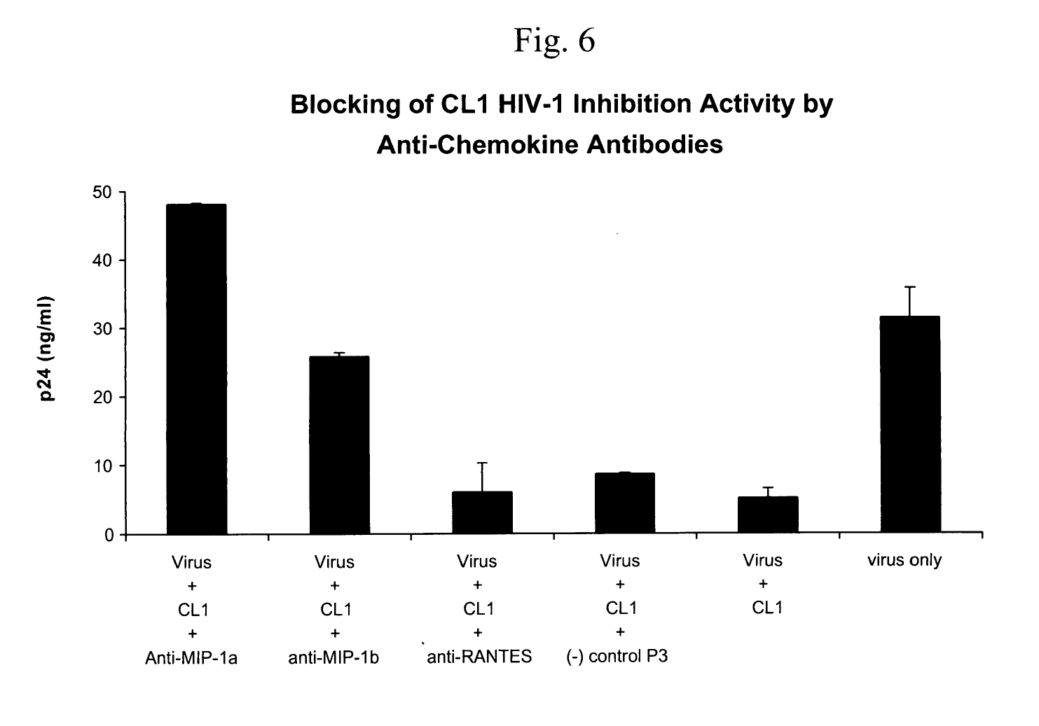

The ability of antibodies against CCR5 chemokines to inhibit the capacity of anti-lipid antibodies to inhibit HIV-1 infection of PBMC was studied. The question presented was whether antibodies that neutralize the effects of CCR5 chemokines, when added to a PBMC HIV-1 infectivity assay, could inhibit the ability of mAb CL1 to inhibit PBMC infection by HIV-1 (FIG. 6). It was found that antibodies that neutralize the CCR5 chemokines MIP-1α and MIP-1β were the strongest inhibitors of the ability of the anti-lipid antibodies to inhibit HIV infectivity. Thus, indeed, the induction of CCR5 chemokines in the presence of HIV-1 by anti-lipid antibodies can inhibit HIV-1 infection of PBMC.

All documents and other information sources cited above are hereby incorporated in their entirety by reference.

TABLE 1
HIV-1 inhibition activity anti-lipid
and HIV-1 MAbs assayed in PBMC.

IC80 vs primary isolates (μg/mL)

| mAb | B.6535 | C.DU123 |
|---|---|---|
| IS4 | 0.07 | 0.06 |
| CL1 | 0.42 | 0.19 |
| P1 | 30 | <0.2 |
| B1 | >50 | >50 |
| B2 | >50 | >50 |
| Tri-Mab | 2.4 | >25 |

Tri-Mab = 2F5, 2G12, IgG1b12

TABLE 2
HIV-1 inhibition activity anti-lipid and
HIV-1 MAbs assayed in TZM-bl cells.

| | ID50 in pseudovirus assay (μg/mL) | | |
|---|---|---|---|
| mAb | B.6535 | B.PVO | C.DU123 |
| IS4 | >50 | >50 | >50 |
| CL1 | >50 | >50 | >50 |
| P1 | >50 | >50 | >50 |
| B1 | >50 | >50 | >50 |
| B2 | >50 | >50 | >50 |
| 4E10 | 2.2 | <2 | <2 |

TABLE 3
Anti-Lipid Antibodies Inhibit R5 HIV-1 Primary Isolates
With Greater Breadth than 2F5, 2G12 and 1b12 MAbs

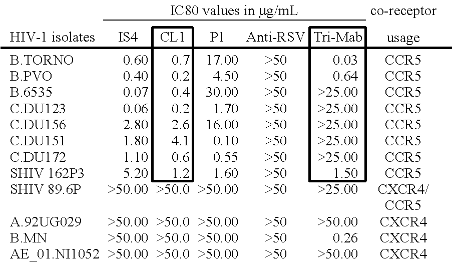

Tri-Mab = 2F5, 2G12, IgG1b12

TABLE 4
HIV-1 Inhibition Activity Assayed in Various Cell Types

| | IC80 values in μg/mL | | | | |
|---|---|---|---|---|---|
| | Mono- | Monocyte | CD4 T | CD4 T cell | |
| mAb | cytes | depleted PBMC | cells | depleted PBMC | PBMC |
| CL1 | 0.06 | >50 | >50 | 14 | >50 |
| 2G12 | 0.17 | 1.46 | 12.3 | 0.4 | 0.2 |

TABLE 5
Inhibition of HIV-1 by anti-Lipid antibodies In PBMC-based
neutralization assays Using LucR-incorporated HIV-1.

| | HIV-1 Isolates | | |
|---|---|---|---|
| | HIV- | HIV- | HIV- |
| Anti- | WITO.LucR. | WITO.LucR. | WEAU3-3.LucR. |
| body | T2A.ecto/hPBMC* | T2A.ecto/hPBMC* | T2A.ecto/hPBMC# |
| IS4 | 0.08 | <0.02 | >50.00 |
| P1 | <0.02 | <0.02 | >50.00 |
| A32 | >50.00 | >50.00 | >50.00 |
| 4E10 | 0.09 | 0.16 | 22.24 |
| ACL4 | 1.00 | 1.33 | >50.00 |
| CL1 | <0.02 | <0.02 | >50.00 |
| Synagis | >50.00 | >50.00 | >50.00 |
| 2F5 | 0.97 | 4.22 | 6.44 |
| 4E10 | 0.05 | 0.33 | 4.52 |

*CCR5 HIV-1 isolate;
#CXCR4 isolate.

What is claimed is:

1. A method of inhibiting infection of susceptible cells of a human subject by a CCR5-tropic strain of HIV-1 comprising administering to said subject an immunogen that induces the production of antibodies that bind to cells of said subject that:
i) produce CCR5-binding chemokines, and
ii) have on their surface an antigen recognized by the antibodies, said immunogen being administered in an amount and under conditions such that, either alone or in the presence of said CCR5-tropic strain of HIV-1, the level of CCR5-binding chemokines produced is sufficient to effect said inhibition of infection of said susceptible cells.

2. The method according to claim 1 wherein said susceptible cells of said subject are T cells.

3. The method according to claim 1 wherein said cells of said subject that produce CCR-5-binding chemokines are monocytes, macrophages or dendritic cells.

4. The method according to claim 1 wherein said antigen is a surface lipid of the cell lipid bilayer.

5. The method according to claim 4 wherein said antigen is phosphatidylserine (PS) or phosphatidylethanolamine (PE).

6. The method according to claim 1 wherein said immunogen comprises an anionic lipid.

7. The method according to claim 6 wherein said anionic lipid is PS, PE, cardiolipin (CL), 1,2-dioleoyl-sn-glycero-3-phosphatidylethanolamine (DOPE), or killed Treponema pallidum.

8. The method according to claim 6 wherein said immunogen comprises a liposome comprising PS, PE or CL.

9. The method according to claim 6 further comprising administering to said subject an adjuvant comprising monophosphoryl lipid A, Toll Like Receptor (TLR)-7 or TLR-9.

10. The method according to claim 1 wherein said immunogen is a hexagonal II form of PS or PE.

11. The method according to claim 1 wherein said antigen is CD40.

12. The method according to claim 11 wherein said immunogen free or derivatized human or rhesus CD40.

13. The method according to claim 12 wherein said CD40 is derivatized with tetanus toxoid or keyhole limpet hemocyanin.

14. The method according to claim 11 wherein said immunogen comprises rhesus, guinea pig or mouse CD40 or mutated form thereof, or a nucleic acid sequence encoding rhesus, guinea pig or mouse CD40 or mutated form thereof, and results in the induction of anti-CD40 antibodies that bind CD40 but not to the CD40Ligand binding site on CD40.

15. The method according to claim 14 wherein said immunogen comprises a nucleic acid sequence encoding rhesus, guinea pig or mouse CD40 or mutated form thereof.

16. The method according to claim 15 wherein said nucleic acid sequence is present in a vector operably linked to a promoter.

17. The method according to claim 1 wherein said antibodies are non-pathogenic.

18. A method of inhibiting infection of susceptible cells of a human subject by HIV-1 comprising administering to said subject an immunogen that induces the production of m43- or m9-type antibodies, said immunogen being administered in an amount and under conditions such that anti-HIV chemokines are produced and said inhibition of infection is thereby effected.

19. A method of inhibiting infection of susceptible cells of a human subject by a CXCR4-utilizing strain of HIV-1 comprising administering to said subject an immunogen that induces the production of antibodies that result in the release of SDF-1 from target cells, said immunogen being administered in an amount and under conditions such that said inhibition is effected.


SEQUENCE LISTING (6 sequences)

SEQ ID NO: 1 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 2 - protein, 278 aa, Macaca mulatta
MVRLPLQCVLWGCLLTAVYPEPPTACREKQYLINSQCCSLCQPGQKLVSDCTEFTETECLPCSESEFLDTWNRETRCHQHKYCDPNLGLRVQQKGTSETDTICTCEEGLHCMSESCESCVPHRSCLPGFGVKQIATGVSDTICEPCPVGFFSNVSSAFEKCRPWTSCETKDLVVQQAGTNKTDVVCGPQDRQRALVVIPICLGILFVILLLVLVFIKKVAKKPNDKAPHPKQEPQEINFLDDLPGSNPAAPVQETLHGCQPVTQEDGKESRISVQERQ

SEQ ID NO: 3 - protein, 289 aa, Mus sp.
MVSLPRLCALWGCLLTAVHLGQCVTCSDKQYLHDGQCCDLCQPGSRLTSHCTALEKTQCHPCDSGEFSAQWNREIRCHQHRHCEPNQGLRVKKEGTAESDTVCTCKEGQHCTSKDCEACAQHTPCIPGFGVMEMATETTDTVCHPCPVGFFSNQSSLFEKCYPWTSCEDKNLEVLQKGTSQTNVICGLKSRMRALLVIPVVMGILITIFGVFLYIKKVVKKPKDNEILPPAARRQDPQEMEDYPGHNTAAPVQETLHGCQPVTQEDGKESRISVQERQVTDSIALRPLV

SEQ ID NO: 4 - protein, 289 aa, Artificial Sequence
  Description of Artificial Sequence Synthetic polypeptide
MVSLPRLCALWGCLLTAVHLGQCVTCSDKQYLHDGQCCDLCQPGSRLTSHCTALEKTQCHPCDSGEFSAQWNREIRCHQHRHCDPNQGLRVKKEGTAESDTVCTCKEGQHCTSEACEACAQHTPCIPGFGVMEMATETTDTVCHPCPVGFFSNQSSLFEKCYPWTSCEDKNLEVLQKGTSQTNVICGLKSRMRALLVIPVVMGILITIFGVFLYIKKVVKKPKDNEILPPAARRQDPQEMEDYPGHNTAAPVQETLHGCQPVTQEDGKESRISVQERQVTDSIALRPLV

SEQ ID NO: 5 - protein, 289 aa, Artificial Sequence
  Description of Artificial Sequence Synthetic polypeptide
MVSLPRLCALWGCLLTAVHLGQCVTCSDKQYLHDGQCCDLCQPGSRLTSHCTALEKTQCHPCDSGEFSAQWNREIRCHQHKYCDPNLGLRVQQKGTSETDTICTCKEGWHCTSEACEACAQHTPCIPGFGVMEMATETTDTVCHPCPVGFFSNQSSLFEKCYPWTSCEDKNLEVLQKGTSQTNVICGLKSRMRALLVIPVVMGILITIFGVFLYIKKVVKKPKDNEILPPAARRQDPQEMEDYPGHNTAAPVQETLHGCQPVTQEDGKESRISVQERQVTDSIALRPLV

SEQ ID NO: 6 - protein, 278 aa, Artificial Sequence
  Description of Artificial Sequence Synthetic polypeptide
MVRLPLQCVLWGCLLTAVYPEPPTACREKQYLINSQCCSLCQPGQKLVSDCTEFTETECLPCSESEFLDTWNRETRCHQHKYCDPNLGLRVQQKGTSETDTICTCEEGLHCMSEACESCVPHRSCLPGFGVKQIATGVSDTICEPCPVGFFSNVSSAFEKCRPWTSCETKDLVVQQAGTNKTDVVCGPQDRQRALVVIPICLGILFVILLLVLVFIKKVAKKPNDKAPHPKQEPQEINFLDDLPGSNPAAPVQETLHGCQPVTQEDGKESRISVQERQ